(19) United States
(12) Patent Application Publication
(10) Pub. No.: US 2024/0418711 A1
(43) Pub. Date: Dec. 19, 2024

(54) NEW NANOPARTICLES FOR USE IN THE DETECTION OF TARGET BIOMOLECULES

(71) Applicant: APLEX BIO AB, Solna (SE)

(72) Inventors: Umear Naseem, Solna (SE); Klaus Kunze, Albuquerque, NM (US)

(21) Appl. No.: 18/698,435

(22) PCT Filed: Oct. 6, 2022
(86) PCT No.: PCT/EP2022/077835

§ 371 Date: Apr. 4, 2024

Related U.S. Application Data
(60) Provisional application No. 63/252,744, filed on Oct. 6, 2021.

(51) Int. Cl. G01N 33/543; G01N 21/64; G01N 33/53

(52) U.S. Cl. CPC G01N 33/54346; G01N 21/6428; G01N 33/5308; G01N 2021/6439

(57) ABSTRACT

The present disclosure relates to a nanoparticle which comprises a core comprising a first fluorophore, preferably a semiconductor, and a first layer comprising a second fluorophore, wherein the emission and/or excitation wavelength of the first fluorophore is different to the emission and/or excitation wavelength of the second fluorophore, along with processes for preparing such a nanoparticle, methods for the detection of target biomolecules using such a nanoparticle, uses of such a nanoparticle and a kit-of-parts comprising such nanoparticle.

### FIELD OF THE INVENTION

The present disclosure relates to a nanoparticle which comprises a core comprising a first fluorophore, preferably a semiconductor, and a first layer comprising a second fluorophore, wherein the emission and/or excitation wavelength of the first fluorophore is different to the emission and/or excitation wavelength of the second fluorophore, along with processes for preparing such a nanoparticle, methods for the detection of target biomolecules using such a nanoparticle, uses of such a nanoparticle and a kit-of-parts comprising such a nanoparticle.

### BACKGROUND

The listing or discussion of an apparently prior-published document in this specification should not necessarily be taken as an acknowledgment that the document is part of the state of the art or common general knowledge.

Many bioanalytical technologies typically use fluorescent methods to allow for multiplexed (more than one) detection of various targets. Several applications benefit from doing this such as histology, flow cytometry, fundamental cellular and molecular protocols, fluorescence in situ hybridization, DNA sequencing, immunoassays, binding assays, nucleic acid profiling (genomics/transcriptomics), protein profiling (proteomics) etc. While it is possible to detect more than one target simultaneously, using fluorophores alone still severely limits the ability to multiplex. For the in situ hybridization application especially, new technologies are emerging in order to increase the magnitude of multiplexing capacity focused on barcode decoding via in situ sequencing schemes.

The use of multiple fluorophores in a single step analysis is limited by their physical properties, where each fluorophore has a certain spectral shape of its absorbance and emission (about 100 nm broad for organic fluorophores). Due to this, when multiple fluorophores are used simultaneously their emissions need to be well separated. This causes a limitation in the number of different fluorophores that can be used simultaneously before the emission overlaps (spectral overlap) such that it becomes difficult to distinguish one fluorophore from another (typically less than 5-10 fluorophores simultaneously).

In order to overcome this, in situ sequencing technologies are emerging. Strell et al. (FEBS J. (2018), p. 14435 (Placing RNA in context and space-methods for spatially resolved transcriptomics)) and Lein et al. (Science (2017) 358, 64-69 (The promise of spatial transcriptomics for neuroscience in the era of molecular cell typing)) are two relevant articles in this context. These make use of molecular DNA barcodes that each represent a unique target that needs to be decoded for the successful detection to be completed (targeted method), or alternatively doing the direct sequencing of the target in case of RNA/DNA (nontargeted). The process of decoding involves the use of DNA sequencing chemistry (addition of signal and removal), the iterative process of stepping one DNA base at a time and repeating this the necessary amount of times depending on the level of multiplexing desired, maintaining the integrity and location of the sample during this iterative process, image acquisition between each iteration, and finally the image analysis to puzzle and align all images together to thereafter decide on the identity of each target. The whole process therefore takes much longer than that of one-step (direct) detection methods (such as conventional fluorescent in situ hybridization), and typically requires trained personnel and/or robust hardware that integrates chemical steps with imaging steps resulting in high costs. Furthermore, the process of doing several steps iteratively also makes the sequencing approach sensitive to errors or mistakes, as it is enough for a sub-part of the process to go wrong for the whole decoding to fail altogether. The sequencing process is, therefore, more complicated, sensitive to errors, expensive, and time consuming in comparison to a direct approach.

It is, however, a challenge to achieve sufficiently high multiplexing nanoparticles (more than 20-100) while retaining a small particle size that enable small target biomolecule detection, especially in-situ but not limited to in tissue or cells. The challenge is to synthesize nanoparticles that are encoded with high enough precision to enable low polydispersity in fluorophore incorporation, the number of different fluorophores that can be used simultaneously in a limited physical space and have high enough emission intensity and photostability to enable the number of color combinations and levels needed to achieve high multiplexity.

There is, therefore, a need for improved fluorophore complexes that allow for improved methods for multiplexed detection of a plurality of target biomolecules.

### DESCRIPTION OF THE INVENTION

The inventors have unexpectedly found an improved fluorophore-nanoparticle complex that mitigates, alleviates or eliminates one or more of the above-identified deficiencies and disadvantages in the prior art and solves at least one of the above-mentioned problems.

Such highly multiplexing nanoparticles, which may be combined with co-labelling of amplified target biomolecules in a co-localized spot, enable a new way to parallelize multiplexed detection to a high degree with state-of-the art sensitivity and specificity of bioassays.

### Nanoparticles According to the First Aspect of the Invention

According to a first aspect of the invention there is provided a nanoparticle which comprises:
a. a core comprising a first fluorophore, which is a semiconductor; and
b. which core is coated with a first layer, wherein the first layer comprises a second fluorophore;
   wherein, the emission and/or excitation wavelength of the first fluorophore is different to the emission and/or excitation wavelength of the second fluorophore.

The nanoparticle of the first aspect of the invention is referred to herein as a core-layer nanoparticle, that is to say that the nanoparticle comprises a central core, for example a single core, of one material being a semiconductor with a layer, or multiple layers, that surround the core, wherein the core and surrounding layers differ either physically (for example in atomic structure) or chemically (for example in the materials that they are composed of).

Other core-layer nanoparticles are described herein under the section titled “Alternative Inventive Nanoparticles”.

When stating that the emission and/or excitation wavelength of the first fluorophore is different to the emission and/or excitation wavelength of the second fluorophore, we are referring to the two fluorophores having different overall optical spectra such that the fluorophores are able to be distinguished from one another by excitation/emission filters.

For the avoidance of doubt, the term “nanoparticle(s)” as used herein is also referred to, interchangeably, as the abbreviation “NP(s)”.

The first fluorophore semiconductor may be selected from the list consisting of quantum dots, rods (such as quantum rods), quantum rods, Pdots, and mixtures thereof.

By the term “rods” we refer to herein elongated particles where length/width ratio is not equal to 1.

By the term “Pdot” as used herein, we refer to semiconducting polymer dots.

The core may comprise a single quantum dot, rod, quantum rod, or Pdot, which act as the first fluorophore. Alternatively, the core may comprise a cluster of quantum dots, rods, quantum rods, or Pdots, which clusters act as the first fluorophore.

Indeed, the core may consist of a single quantum dot, rod, quantum rod, or Pdot. Alternatively, the core may consist of a cluster of quantum dots, rods, quantum rods, or Pdots.

By the term “quantum dot”, we refer to semiconductor particles a few nanometres in size, typically in the range of from about 2 to about 10 nm, having optical and electronic properties that differ from larger particles. The term “quantum dot” is also referred to herein by the abbreviation QD throughout.

The quantum dot(s) used as the first fluorophore may be selected from the list consisting of semiconductor quantum dots, perovskite quantum dots, and silicon quantum dots.

The semiconductor quantum dots may be quantum dots that are alloys containing elements from groups III and V of the periodic table, groups IV and VI of the periodic table, or groups II and VI of the periodic table.

For example, the semiconductor quantum dots may comprise, or be composed of, PbS, CdSe, CdS, CdSe/CdS, CdSe/ZnS, CdSeS/ZnS, InP, InP/ZnS, and mixtures thereof. The quantum dots may also be core/shell/shell and core/shell/shell/shell quantum dots, for example CdSe/ZnS/CdZnS/ZnS quantum dots.

Additionally, the semiconductor quantum dots may comprise a shell composed of a perovskite material.

The perovskite quantum dots may have the general formula ABX₃, wherein A may be selected from the group consisting of methyl ammonium, formamidinium, Cs, Rb and K; B may be selected from the list consisting of Pb, Cd, Zn, Sn, In, Fe and Sb; and X may be a halogen, preferably selected from the list consisting of Cl, Br and I.

The perovskite quantum dots may also be alloyed versions of perovskite QDs where atoms on the B site are partially replaced by another element, for example CsPb_{1-y}M_{y}X₃ (M=for example Mn, Ni, Sr, Sn).

The perovskite quantum dots may comprise a shell composed of a metal oxide, a perovskite material that is different to the core perovskite material, a metal chalcogenide, a metal organic framework or a polymer.

For example, the perovskite quantum dots may be CsPbBr₃ perovskite quantum dots with an SiO₂ shell, or CsPbBr₃ quantum dots with an SiO₂ first shell and a poly(ethylene glycol) second shell deposited thereon.

The silicon quantum dots may comprise a water soluble coating, such as poly(acrylic acid) or allylamine. It is to be noted that silicon-based quantum dots are environmentally safer and more biocompatible than Cd containing quantum dots and are, therefore, safer to use.

The core of the nanoparticle may have a size in the range of from about 2 to about 150 nm, for example about 2 to about 100 nm, such as about 2 to about 60 nm, for example about 2 to about 50 nm, such as about 2 nm to about 10 nm, or from about 50 to about 150 nm, such as from about 70 to about 130 nm.

The first layer that coats the core may comprise a matrix in which the second fluorophore is embedded. As used herein, when referring to fluorophores being embedded in a matrix, this means that the fluorophores are incorporated into the bulk mass structure of that portion of the nanoparticle in question. For the avoidance of doubt, this does not mean that some of the fluorophore molecules cannot also be on the surface, so long as some of the molecules are in the bulk.

Alternatively, the first layer may be composed of the second fluorophore alone. That is to say, the core may be coated with the second fluorophore which forms the first layer upon which a second layer is deposited. The second layer preferably comprises a matrix as defined above and may also comprise a further fluorophore which is different to the first and second fluorophore.

The second fluorophore may be a metal-organic fluorophore, a semiconductor, an inorganic fluorophore, an organic fluorophore, or combinations thereof.

Particular metal-organic fluorophores for use as the second fluorophore may be metal-ligand complexes (MLCs) such as luminescent terbium complexes (LTCs) or luminescent Eu complexes. Such complexes are advantageous as their emission spectra are multicolored with sharp emission peaks. Another advantage of MLC fluorophores is their robust photochemical stability in particular under physiological conditions.

Near-infrared (NIR) emissive metal complexes may also be used as the second fluorophore. Such complexes possess distinct advantages such as low background and minimal damage to biological tissues. Examples are metal ligand complexes emitting from the ligand such as dicyanomethylene-benzopyran (DCMB) based phenol-bridged dinuclear Zn(II)-DPA (DPA: Dipicolylamine); and metal ligand complexes emitting from the metal centers such as Ru—Gd complexes bridged by N-heterocyclic ligands.

Furthermore, metal organic frameworks (MOFs) may be used as metal-organic fluorophores. Luminescent properties are largely determined by the fine tuning of their emission profiles through modulating interaction between organic linker components, the choice of luminescent metal ions and encapsulation of further fluorophores.

When the second fluorophore is a semiconductor, this may be selected from the list consisting of quantum dots, rods, quantum rods, Pdots, and mixtures thereof.

Inorganic fluorophores that may be used as the second fluorophore may be selected from the list of up converting (UCNPs), noble metal dots, and combinations thereof.

The quantum dot(s) used as the second fluorophore may be selected from the list consisting of semiconductor quantum dots, perovskite quantum dots, silicon quantum dots, carbon quantum dots, Pdots, and combinations thereof.

For the second fluorophore, where the class of potential fluorophores to use overlaps with that of the first fluorophore, the second fluorophore may be selected to be any of the specific fluorophores outlined for the first fluorophore.

Particular organic fluorophores that may be used as the second fluorophore can be selected from the list consisting of Atto 425, Alexa fluor 405, Alexa Fluor 488, fluorescine, DiO, Atto 488, BODIPY FL, Cy3, DiI, Alexa fluor 546, Atto 550, BODIPY TMR-X, Cy5, Alexa fluor 647, Texas red, DiD, Atto647(N), Atto 655, Cy7, Alexa fluor 680, Alexa fluor 750, Atto 680, and Atto 700, BODIPY, Brilliant Violet, Cyanine, Alexa, Atto, fluorescein, coumarin, rhodamine, xanthene fluorophore families and derivatives and combinations thereof.

Further details regarding the particular organic fluorophores that may be incorporated into the first layer of the nanoparticle are provided in the table below.

| Fluorophore | |
|---|---|
| Colour Type | Fluorophore Generic Name and IUPAC Name |
| Blue | Atto 425 |
| | 4-[3-(ethoxycarbonyl)-6,8,8-trimethyl-2-oxo-7,8-dihydro-2H-py- |
| | rano[3,2-g]quinolin-9(6H)-yl]butanoic acid |
| | Alexa fluor 405 |
| | tris(N,N-diethylethanaminium) 8-[2-(4-{[(2,5-dioxopyrrolidin-1- |
| | yl)oxy]carbonyl}piperidin-1-yl)-2-oxoethoxy]pyrene-1,3,6-trisul- |
| | fonate |
| Green | Alexa fluor 488 |
| | 6-amino-9-(2,4-dicarboxyphenyl)-4,5-disulfo-3H-xanthen-3-iminium |
| | Fluorescein |
| | 3′,6′-dihydroxyspiro[isobenzofuran-1(3H),9′-[9H]xanthen]-3-one |
| | DiO |
| | 3,3′-Dioctadecyloxacarbocyanine Perchlorate |
| | Atto 488 |
| | 4-{1-[2-(6-amino-3-imino-4,5-disulfo-3H-xanthen-9- |
| | yl)phenyl]-N-methylformamido}butanoic acid |
| | BODIPY FL |
| | 3-{2-[(3,5-Dimethyl-1H-pyrrol-2-yl-κN)methylene]-2H-pyrrol-5-yl- |
| | κN}propanoato)(difluoro)boron |
| Yellow - Orange | Cy3 (amine and derivates) |
| | 6-[6-[(2E)-3,3-dimethyl-2-[(E)-3-(1,3,3-trimethylindol-1-ium-2- |
| | yl)prop-2-enylidene]indol-1-yl]hexanoylamino]hexylazanium; |
| | dichloride |
| | DiI |
| | 1,1′-Dioctadecyl-3,3,3′,3′-Tetramethylindocarbocyanine |
| | Alexa fluor 546 |
| | 2,3,5-trichloro-4-{[({6-[(2,5-dioxopyrrolidin-1- |
| | yl)oxy]-6-oxohexyl}carbamoyl)methyl]sulfanyl}-6- |
| | (2,2,4,8,10,10-hexamethyl-12,14-disulfo- |
| | 2,3,4,8,9,10-hexahydro-1H-13-oxa-1,11- |
| | diazapentacen-6-yl)benzoic acid |
| | Atto 550 |
| | 4-{1-[2-(2,2,4,8,10,10-hexamethyl- |
| | 2,3,4,8,9,10,11,11a-octahydro-1H-13-oxa-1,11- |
| | diazapentacen-6-yl)phenyl]-N- |
| | methylformamido}butanoic acid |
| | BODIPY TMR-X |
| | [N-{6-[(2,5-dioxopyrrolidin-1-yl)oxy]-6-oxohexyl}-3-(2-{[5-(4- |
| | methoxyphenyl)-1H-pyrrol-2-yl-kappaN]methylene}-3,5-dimethyl- |
| | 2H-pyrrol-4-yl-kappaN)propanamidato](difluoro)boron |
| Red | Cy5 |
| | 6-[6-[(2E)-3,3-dimethyl-2-[(2E,4E)-5-(1,3,3-trimethylindol-1-ium- |
| | 2-yl)penta-2,4-dienylidene]indol-1-yl]hexanoylamino]hexylazanium |
| | Alexa fluor 647 |
| | 2-[5-[3,3-dimethyl-5-sulfo-1-(3-sulfopropyl)indol-1-ium-2-yl]penta- |
| | 2,4-dienylidene]-3-methyl-3-[5-oxo-5-(6-phosphonooxyhexyla- |
| | mino)pentyl]-1-(3-sulfopropyl)indole-5-sulfonic acid |
| | Texas Red |
| | 5-chlorosulfonyl-2-(3-oxa-23-aza-9-azoniaheptacy- |
| | clo[17.7.1.15,9.02,17.04,15.023,27.013,28]octacosa- |
| | 1(27),2(17),4,9(28),13,15,18-heptaen-16-yl)benzenesulfonate |
| | DiD |
| | 1,1′-Dioctadecyl-3,3,3′,3′-Tetramethylindodicarbocyanine |
| | Atto 647(N) |
| | 14-{2-[(3-carboxypropyl)(methyl)carbamoyl]phenyl}-7- |
| | ethyl-3,3,8,8,10-pentamethyl-7,21lambda5- |
| | diazahexacyclo[15.7.1.0^{2,15}.0^{4,13}.0^{6,11}.0^{21,25}]pentacosa- |
| | 1,4(13),5,11,14,16,21(25)-heptaen-21-ylium |
| | Atto 655 |
| | 4-[1-ethyl-10,10-dimethyl-8-(sulfomethyl)- |
| | 3,4,8,9,10,11-hexahydro-2H-13-oxa-1lambda4,6,11- |
| | triazapentacen-11-yl]butanoic acid |
| Near-IR | Cy7 |
| | 1-(5-carboxypentyl)-2-[7-(1-ethyl-5-sulfo-1,3-dihydro-2H-indol-2- |
| | ylidene)hepta-1,3,5-trien-1-yl]-3H-indolium-5-sulfonate |
| | Alexa fluor 680 |
| | N/A |
| | Alexa fluor 750 |
| | N/A |
| | Atto 680 |
| | 1-(3-carboxypropyl)-11-ethyl-2,2-dimethyl-4- |
| | (sulfomethyl)-2,8,9,10-tetrahydro-1H-13-oxa-1,6,11- |
| | triazapentacen-11-ium |
| | Atto 700 |
| | N/A |

The UCNPs may have the general formula NaYF₄:RE1, RE2 or YF₃:RE1, RE2 or Gd₂O₃:RE1, RE2 where RE1 and RE2 are independently selected from the list of Er, Yb, Tm, Ho. Examples are NaYF₄:Yb, Er; Gd₂O₃:Yb, Er; NaYF₄:Yb, Tm. Examples for incorporation of UCNP into core shell structures are NaYF₄:Yb, Er/Ag and Ag/SiO₂/Lu₂O₃:Gd, Yb, Er. UCNPs are advantageous for bioimaging as they do not exhibit interference from autofluorescence (important for in situ application), and they do not exhibit photobleaching.

The noble metal dots may be Au or Pd metal dots.

By using semiconductor fluorophores, such as quantum dots, in the core this allows to achieve a greater range of combinations with other fluorophores in the layer(s) e.g. metal-organic fluorophores, semiconductors, and/or organic fluorophores due to: (1) having different, non-overlapping, optical (absorbance & emission) spectra than organic fluorophores or metal-organic fluorophores; and (2) having a narrow emission spectra that enables using multiple colors without overlap with metal-organic fluorophores, semiconductors, and/or organic fluorophores.

To explain further, the number of combinations becomes n^{m-1}, where n is the number of intensity levels and m the number of colors. It follows that the number of combinations scales greater with the number of colors rather than the number of intensity levels. Using semiconductor fluorophores as the core it is possible to achieve both a narrower emission spectrum (enabling more colors in parallel before spectral overlap becomes a problem), as well as fitting in more colors in a spectrum together with organic fluorophores due to the possibility of having semiconductor fluorophores with very large stoke shifts that do not overlap with the spectrum of organic fluorophores (meaning that at least the emission or absorbance or both is separated between the fluorophores).

In particular, by using semiconductor fluorophores (such as quantum dots) as seeds for layer growth, a method for synthesizing core-layer nanoparticles can be established that ensures only one semiconductor (e.g. one quantum dot) incorporation per nanoparticle. This allows for an even greater range of multiplexing combinations with other fluorophores because the core fluorophore can act as an internal reference towards the other fluorophores in the core-layer nanoparticle. The property of having the core as an internal reference together with the above-mentioned properties of non-overlapping spectrums and wider range of colors impacts the range of combinations as can be described as q·n^{m-1}, where n is the number of intensity levels, m the number of colors from the second fluorophores, and q the number of colors from the core which are non-overlapping colors with the second fluorophores (m), such as the number of different QDs.

In particular, the internal reference is important in the method of probing a target biomolecule with the nanoparticles where the number of particles bound to the target biomolecule is unknown. If such internal reference is missing, not all combinations according to n^{m-1} can be used since the intensity levels (m) are not only a function of the fluorophore encoding, but also of how many nanoparticles are bound to the biomolecule.

In an embodiment, the first fluorophore and the second fluorophore are incorporated into the nanoparticles in predetermined ratios so that on excitation the nanoparticles exhibit a unique emission spectrum, i.e. the emission wavelength and intensity from the nanoparticle is controlled. That is to say, the precise incorporation of the first and second fluorophores into the nanoparticles enables them to be optically encoded, meaning that they are particularly adept for use in methods of simultaneously detecting a significant plurality (e.g. about 50 to about 100) of detection targets in situ.

As used herein, the term “a plurality” refers to at least two.

Put in another way, by controlling the ratios of the first and second fluorophores to one another, this arrives at a nanoparticle that exhibits at least two specific emission wavelengths and intensities that is unique to the nanoparticle.

When referring to ratios of the first and second fluorophore as used herein we include reference to the fluorophores being incorporated in the nanoparticles in a predetermined amount. The ratio may be any type of ratio, such as weight ratio, atomic ratio or volume ratio.

For the avoidance of doubt, although the second fluorophores may also be selected to be a semiconductor, this will be different to the semiconductor of the core so that the emission and/or excitation wavelength of the first fluorophore is different to the emission and/or excitation wavelength of the second fluorophore.

By using nanoparticles it is also possible to alter the properties of individual fluorophores when incorporated in an organized matter in a particle where distance between the fluorophores can be carefully controlled, such that they can be coupled energetically and act as waveguides/antennas, such as Förster resonance energy transfer (FRET) or excitation energy transfer (EET). This allows for further flexibility in achieving a higher number of concentration levels and/or higher number of color combinations, leading ultimately to a higher number of multiplexity.

Furthermore, the metal-organic fluorophores can be lanthanide complexes, such as luminescent europium complexes or luminescent terbium complexes (LTC). Such fluorophores exhibit properties such as sharp emission peaks, multiple emission peaks (multi-color emission) and long stoke-shifts. These properties, alone or in combination, can be used to further expand the number of colors that can be used simultaneously in the multiplexing nanoparticle. For example, even if the emission of LTCs overlap with the emission of the other fluorophores in the nanoparticle, because the emission from the LTC is multipeak emission the signal can be deconvoluted from the other fluorophores by smart algorithms. Furthermore, filter setups can be arranged such that at least one of the peaks of the LTC can be independently measured, thereby deconvoluting the emission from the other potentially overlapping peaks.

The nanoparticles can, in addition, provide protection to the organic fluorophores to prevent bleaching. This is important because the number of combinations becomes limited if the intensity distributions overlap with each other.

For example, by encapsulating organic fluorophores in the matrix of the first layer of the nanoparticles, the process of bleaching of the fluorophores can be slowed down. For example, bleaching proceeds faster in the presence of oxygen, so slowing the diffusion of oxygen into the particle is one way to slow bleaching. The other aspect is that the rate of photobleaching is dependent on the environment, for example solvent conditions. This way, the “solvent conditions” inside the particle can be made different from outside the particle, for example when encapsulating the fluorophores in a non-polar matrix such as a polymer matrix. Although the benefits of encapsulation with regard to bleaching have been explained above for organic fluorophores, the advantages also apply when using other fluorophores as bleaching also occurs in non-organic fluorophores. For example, unwanted blinking from the QD core can also be controlled through the presence of the first layer.

The first layer may have a thickness of from about 1 nm to about 100 nm, such as from about 5 to about 50 nm, for example about 5 to about 30 nm.

The nanoparticle may comprise additional layers to the first layer, such as a second layer, a third layer, a fourth layer and/or a fifth layer, wherein each additional layer may comprise a further fluorophore which can be any fluorophore outlined herein.

The nanoparticle may comprise further fluorophores, such as a third, fourth, fifth, sixth, seventh, eighth, ninth and/or tenth fluorophore and these may be incorporated into the core, the first layer, or any of the further layers. For example, the nanoparticle may comprise a total of 5 to 8 fluorophores, which includes both the semiconductor first fluorophore and the second fluorophore. The inventors have found that nanoparticles comprising 5 to 8 fluorophores maximizes the number of fluorophores that can be spectrally resolved when taking into account optical spectrums.

The further fluorophore(s) may be any fluorophore as defined herein. As with the first and second fluorophore, the further fluorophores may be incorporated into the nanoparticles in predetermined ratios with respect to the rest of the fluorophores in the nanoparticle so that on excitation the nanoparticles exhibit a unique emission spectrum, i.e. the emission wavelength and intensity from the nanoparticle is controlled.

With the core of the nanoparticle being a quantum dot, this allows to provide the nanoparticle with a constant signal intensity acting as an internal reference and, when including multiple further fluorophores, the ratio of these fluorophores can be altered as needed whilst keeping the intensity signal constant from the core, thus arriving at highly tunable multiplexing nanoparticles that allow for a larger number of combinations to be achieved.

For the avoidance of doubt, the further fluorophores may be contained within their own layers, or each layer may comprise multiple fluorophores. For example, the first layer may comprise at least two fluorophores, where the ratios of these fluorophores can be tuned to achieve different emission signatures.

The use of further fluorophores in either the first layer, or within further layers, allows for an increased number of combinations to be achieved.

In an embodiment, the core consists of a first fluorophore which is a semiconductor, and a first layer which is a matrix, e.g. a silica or polymer matrix, comprising two additional fluorophores (fluorophore two and three) which are organic or organometallic embedded in the matrix. Furthermore, an additional layer which is a matrix, e.g. a silica or polymer matrix, with a fourth fluorophore which is organic or organometallic is added in the matrix. By separating the fourth fluorophore from the second and third, it can be ensured that each fluorophore can be incorporated in high enough concentrations in the matrix without inducing FRET effects which otherwise would affect the emission properties of the fluorophores and thereby affect the encoding. This happens when fluorophores with spectral overlap are in close proximity to each other. In other words, by physically separating the fluorophores from each other by introducing layering and thereby a physical distance, it is possible to reduce FRET effects as FRET is strongly proportional to the distance between donor/acceptor coupled fluorophores.

The thickness of the additional layers may be from about 1 nm to about 100 nm, such as from about 5 to about 50 nm, for example about 5 to about 30 nm.

The nanoparticle may comprise an outer layer. That is to say, the outer layer is positioned so that it is the outermost layer of the nanoparticle, with the first layer, and any subsequent layers (e.g., a second, third, fourth, or fifth layer) being positioned between the core and the outer layer, but it is to be understood that this outer layer could comprise a coating as defined herein.

The outer layer may comprise a metal, such as gold or silver. Such a layer allows for enhanced fluorescence and also acts as a protective layer towards photobleaching.

Metal based objects, including core objects or shells, exhibit a phenomenon called surface plasmon resonance where incident light is converted strongly into electron currents. Nanostructures furthermore are so small that they exhibit quantum mechanical effects that allow them to interact strongly with light waves despite the wavelength of the light being much larger than the nanostructure. Metal based plasmonic nanoparticles therefore produce sub-wavelength confinement and enhancement of optical fields. This effect has been shown to significantly improve the emission properties of fluorophores by affecting the absorption cross section, faster radiative decay, stabilizing blinking in semiconductors and higher quantum efficiency of the fluorophores in optimal proximity to the metallic layer. This effect, similar to surface enhanced raman scattering, is known as surface enhanced fluorescence.

Photobleaching occurs due to higher fluorophore chemical reactivity when in an excited state. This degradation occurs due to photochemical reactions that often involves molecular oxygen and is coupled with the production of singlet oxygen. A metallic outer layer can provide a layer which prevent or slows down the diffusion of molecular oxygen to the inside of the particle, thereby promoting an enhanced photostability for the incorporated fluorophores. Such photostability is an important issue when it comes to encoding of the particles, as the number of combinations of pre-determined concentrations of fluorophores is dependent on how well separated these distributions are to each other. Significant photobleaching can result in large and less separated emission distributions of the fluorophores, significantly limiting the number of levels that can be achieved according to above formula describing the theoretical number of combinations possible.

The outer layer may comprise a hydrophobic polymer, which acts as a protective layer and/or provide colloidal stability in solution. The hydrophobic polymer layer may also serve as anchor for further functionalization of the nanoparticles. Suitable hydrophobic polymers that may be incorporated into the outer layer may be selected from the list consisting of poly(methyl methacrylate), polystyrene, poly(lactic-co-glycolic acid)-azide, poly(lactic-co-glycolic acid)-polyethylene glycol-azide, poly(N-isopropylacrylamide), poly lactic acid, poly-L-lysine, chitosan, dextran-poly(ε-caprolactone), polyacrylic acid-polystyrene, and combinations thereof.

When the first, or subsequent, layer(s) surrounding the core are a polymer matrix, the polymer matrix may comprise or be composed of a hydrophobic polymer, such as a polymer selected from the list consisting of poly(methyl methacrylate), polystyrene, poly(lactic-co-glycolic acid)-azide, poly(lactic-co-glycolic acid)-polyethylene glycol-azide, poly(N-isopropylacrylamide), poly lactic acid, poly-L-lysine, chitosan, dextran-poly(ε-caprolactone), polyacrylic acid-polystyrene, and combinations thereof.

The outer layer may comprise an inorganic oxide or hydroxide, such as silicon oxide, zinc oxide, manganese oxide (MnO₂), cobalt oxide hydroxide (CoOOH) and combinations thereof.

The outer layer may have a thickness of from about 1 nm to about 25 nm, such as about 1 to about 15 nm, for example about 1 to about 10 nm.

The nanoparticle may comprise a coating, wherein the coating comprises a repulsive component, a detection probe, a linker, or combinations thereof.

For the avoidance of doubt, the coating, when present, is on the outermost layer of the nanoparticle, which may be the first layer, any of the further layers, or the outer layer as defined above.

The term “detection probes” refers to molecules that bind specifically to a target biomolecule or a group of target biomolecules. Suitable detection probes may be selected from the list consisting of oligonucleotides with deoxyribose and/ribose bases, xeno nucleic acid (such as locked nucleic acids (LNA)), glycol nucleic acids (GNA), threose nucleic acids (TNA), phosphoroiamidate Morpholino oligomers (PMO), peptide nucleic acids (PNA), antibodies, antibody fragments, synthetic peptides, aptamers, DARPins and combinations thereof.

By the term “repulsive component” this refers to a component that when present on the surface of the nanoparticle provides stability to the nanoparticle. For example, when a plurality of nanoparticles are in solution, the repulsive component prevents the nanoparticles from agglomerating. The stability may be provided by electrostatic interactions or by steric interactions.

The repulsive component may be a charged group with a positive or negative charge, a zwitterionic group, a sterically repulsive group such as polymer chain or an aliphatic chain.

The repulsive component may be a component selected from the group consisting of carboxylic acids, amines, ammonium cations, phosphonic acids, silanes, organosilanes, sulfonic acids, phosphines, hydroxyls, catechols, gallols, or molecules comprising combinations thereof. Such groups are either charged, or exhibit a charge on change of pH when in solution.

The repulsive component may be a sterically repulsive group, such as polymer chain optionally selected from the list consisting of poly(ethylene glycol)/poly(ethylene oxide), poly(propylene glycol), polypeptide, polyglycerol and polyoxazolines, polyacrylamide, poly(acrylic acid), poly(methyl methacrylate and poly (methyl acrylate), and combinations thereof.

Furthermore, the sterically repulsive group may be a C₂ to C₁₈ aliphatic chain, such as a C₆ to C₁₈ aliphatic chain. For example, the aliphatic chain may be selected from the group consisting of hexane, decane, pentadecane, octadecane, polyacetylene, polystyrene and polyethylene, and combinations thereof.

The polymer and/or aliphatic chain may have a molecular weight of less than about 4000 Da, such as from about 150 to about 4000 Da, such as from about 150 to about 2000 Da, for example from about 150 to about 1000 Da.

It is preferable that the polymers are of a molecular weight that provides stability to the nanoparticles in solution through steric stability, but still allows for the coating to incorporate further functionalities, such as detection probes. Therefore, the coating may further, or alternatively, comprise at least one or a plurality of detection probes, optionally selected from the list consisting of a nucleic acid molecule, an antigen, an antibody or combinations thereof.

When the coating comprises a repulsive component and a detection probe, this allows for the multiple nanoparticles in solution together to repulse each other and other surfaces to form a stable dispersion while still being able to form specific attractive bonds with a detection target (DNA/RNA or antibodies) resulting in that the nanoparticles attaching and immobilizing on a detection target. Efficient attachment happens when the repulsive forces are balanced with the attractive forces of the probe.

The repulsive component(s) and/or detection probe(s) may be connected to the surface of the nanoparticle via the use of linkers.

The linker(s) may be bound to the surface of the nanoparticle and comprise a conjugating group, which is configured to conjugate to detection probe and/or repulsive component.

The conjugating groups may be selected from the list consisting of azides, alkynes, Cyclooctines (Dibenzocyclooctyne (DBCO), trans-cyclooctene (TCO)), Cyclononyne (bicyclo[6.1.0]nonyne (BCN)), tetrazines, avidin, streptavidin, neutrAvidin, biotin, isothiocyanates, isocyanates, sulfonyl chlorides, aldehydes, carbodiimides, acyl azides, anhydrides, fluorobenzenes, carbonates, NHS esters, imidoesters, epoxides, fluorophenyl esters, phosphines, carboxylic acids, maleimides, Haloacetyls (Br—/I—), pyridyl disulfides, thiosulfonates, vinylsulfones, alkoxyamines and hydrazides, and combinations thereof.

The nanoparticle may comprise a plurality of linkers having a conjugating group, such as a conjugating group above, that is not bound to any further moieties (such as detection probes or repulsive components). This design allows for rapid and custom modification of the nanoparticles with various targets to easily create new panels as wished for by the end user.

In an embodiment, the linker is not present meaning that the detection probes and/or repulsive components are bound directly to the nanoparticle surface.

The linker may comprise a spacer group, which may be selected from the list consisting of bioinert polymers, nucleotide oligomers/polymers, hydrocarbons, functional hydrocarbons, and combinations thereof.

Suitable bioinert polymers include poly(ethylene oxides)/poly(ethylene glycols), polypeptides, polyglycerols, polyoxazolines, and combinations thereof.

Suitable nucleotide oligomers/polymers include non-specific or repeat oligonucleotide sequences.

Suitable hydrocarbons include hexane, decane, pentadecane, octadecane, polyacetylenes, polystyrenes, polyethylenes, and combinations thereof.

Suitable functional hydrocarbons include polyacrylamides, poly(acrylic acids), poly(methyl methacrylates), Poly(methyl acrylates), and combinations thereof.

The linker, detection probe and/or repulsive component may be bound to the surface of the nanoparticle, either by conjugation or electrostatic forces, via an anchor (functional group) selected from the list consisting of thiols, aldehydes, disulfides, carboxylic acids, amines, azydes, alkynes, cycloocctines (e.g. dibenzocyclooctyne (DBCO) and trans-cyclooctene (TCO)), ammonium cations, cyclononyne (e.g., bicyclo[6.1.0]nonyne (BCN)), phosphonic acids, silanes, organosilanes, sulfonates, phosphines, hydroxyls, catechols, gallols, ethoxysilanes, methoxysilanes, tetrazines, silazanes, chlorosilanes, avidin, streptaviding, neutravidin and biotin.

When the outer layer of the nanoparticle is comprised of a metal (e.g., gold), the linker, detection probe and/or repulsive component may be bound to the surface of the nanoparticle via an anchor (functional group) selected from the list consisting of thiols, disulfides, carboxylic acids, amines, ammonium cations, phosphonic acids, silanes, organosilanes, sulfonates, phosphines, hydroxyls, catechols, and gallols.

By the term “anchor” this refers to a group which tethers the coating to the nanoparticle and optionally a spacer group. An anchor group “tethering” the coating to the nanoparticle is to be interpreted as that the anchor group binds the coating to the nanoparticle by covalent or non-covalent binding (e.g., by conjugation or electrostatic forces).

When the outer layer of the nanoparticle is comprised of an organic oxide (e.g., silicon oxide) the linker, detection probe and/or repulsive component may be bound to the surface of the nanoparticle, either by conjugation or electrostatic forces, via an anchor (functional group) selected from the list consisting of ethoxysilanes, methoxysilanes, silazanes and chlorosilanes.

In an embodiment, the diameter of the nanoparticle is less than about 1000 nm, for example less than about 500 nm or about 300 nm, or less than about 100 nm such as from about 3 to about 300 nm, for example, from about 3 to about 200 nm, about 3 to about 150 nm, such as about 100 to 150 nm, or about 3 to about 100 nm.

The nanoparticle size may be measured by any method known in the art, such as transmission electron microscopy (TEM), scattering electron microscopy (SEM) size exclusion chromatography (SEC) or dynamic light scattering (DLS).

Nanoparticles with a size smaller than about 300 nm, and preferably less than about 100 nm are particularly useful in multiplexed detection methods when the sample being analysed comprises single cells or tissue as these sizes ensure for penetration into the cell and/or tissue matrix and that the method can be performed in situ. For the particle to bind to the detection target within the cell and tissue matrix a smaller size is typically advantageous.

In an embodiment, the nanoparticle comprises one or more molecular probes on its surface, each molecular probe comprising a fluorophore that is bound to a nucleic acid molecule, an antigen or an antibody providing binding affinity of the molecular probe to the specific detection target.

In an embodiment the nanoparticle is in solution. Alternatively the nanoparticle may be in a dry, solid, form for dispersion in solution prior to use.

### Alternative Inventive Nanoparticles

According to a second aspect of the invention, there is provided a nanoparticle which comprises;
a. a core comprising a first fluorophore;
b. which core is coated with a first layer, wherein the first layer comprises a second fluorophore; and
c. an outer layer which comprises a metal, an inorganic oxide or a polymer
   wherein, the emission and/or excitation wavelength of the first fluorophore is different to the emission and/or excitation wavelength of the second fluorophore.

According to a third aspect of the invention, there is provided a nanoparticle which comprises;
a. a core comprising a first fluorophore;
b. which core is coated with a first layer, wherein the first layer comprises a second fluorophore; and
c. which core and a first layer is coated with a second layer, wherein the second layer comprises a third fluorophore; and
   wherein, the emission and/excitation wavelengths of the fluorophores are different to each other.

In both the second and third aspect of the invention, the first fluorophore in the core may be a metal-organic fluorophore, an organic fluorophore, an inorganic fluorophore or combinations thereof.

The further layers in the nanoparticles according to the second and third aspect of the invention may comprise any of the features as outlined above for the first aspect of the invention. That is to say, the first aspect of the invention is concerned with nanoparticles having a core comprising a semiconductor first fluorophore, whereas the cores of the nanoparticles comprise a metal-organic fluorophore, an organic fluorophore, an inorganic fluorophore or combinations thereof.

The core in the nanoparticles of both the first and second aspect of the invention may comprise two fluorophore which are organic or organometallic embedded in a silica or polymer matrix, and a first layer comprising one or two additional fluorophores embedded in a silica or polymer matrix.

Particular organic fluorophores that may be incorporated into the core of the nanoparticle as the first fluorophore in the second and third aspect of the invention can be selected from the list consisting of Atto 425, Alexa fluor 405, Alexa Fluor 488, fluorescine, DiO, Atto 488, BODIPY FL, Cy3, DiI, Alexa fluor 546, Atto 550, BODIPY TMR-X, Cy5, Alexa fluor 647, Texas red, DiD, Atto647(N), Atto 655, Cy7, Alexa fluor 680, Alexa fluor 750, Atto 680, and Atto 700, BODIPY, Brilliant Violet, Cyanine, Alexa, Atto, fluorescein, coumarin, rhodamine, xanthene fluorophore families and derivatives and combinations thereof.

The core of the nanoparticles in the second and/or third aspect of the invention may be comprised of a matrix in which the first fluorophore is embedded. For example, the matrix of the core of the nanoparticle may be a polymer matrix, such as a matrix composed of polymers selected from the list consisting of poly(methyl methacrylate), polystyrene, poly(lactic-co-glycolic acid)-azide, poly(lactic-co-glycolic acid)-polyethylene glycol-azide, poly(N-isopropylacrylamide), poly lactic acid, poly-L-lysine, chitosan, dextran-poly(ε-caprolactone), polyacrylic acid-polystyrene.

Alternatively, the matrix of the core of the nanoparticle may be an inorganic oxide matrix, such as a matrix comprising a silicon oxide, zinc oxide, manganese oxide (MnO₂), cobalt oxide hydroxide (CoOOH) and combinations thereof.

### Processes for Preparing the Nanoparticle

According to a fourth aspect of the invention there is provided a process for preparing the nanoparticle of the first aspect of the invention.

In an embodiment, a multilayer particle is synthesized by first providing a core particle acting as a seed for the growth of a first layer. The core particle can be synthesized separately or be used as purchased, such as a quantum dot (core or core/shell semiconductor particle with one or multiple shells). In order to facilitate a seeded growth of a layer around the core particle, initiators for layer growth can be used. Such initiators could be radical initiators attached to the surface of the core particle for initiating polymerization. They can also be monomers or polymers that facilitate adhesion of the monomers used for building the layer matrix (such as tetraethyl orthosilicate in the case of a silica matrix) such as poly-vinylpyrrolidone.

Examples of chemistries includes silica chemistry to form a first silica layer around the core particle, or atom transfer radical polymerization (ATRP) chemistry to form a first polymer layer. Such chemistries initiate the growth from the surface of the particle and can, therefore, be utilized to achieve a high yield of core/layer particles compared to unreacted core particles. Furthermore, it can be ensured that only one core particle is incorporated inside the core/layer particle. This aspect is important when the core fluorophore is going to be used as a reference signal towards the other fluorophores in the particle.

Thus, chemistries such as inorganic silica chemistry and polymer chemistry may be utilized to form layers.

Such chemistries furthermore allow the incorporation of other fluorophores into the matrix, enabling a multi-compartment encoded nanoparticle where the number of fluorophores incorporated are pre-determined and controlled during synthesis.

In another embodiment, an additional outer layer can be grown onto the core/layer particle, where the layer acts as a protective layer and when noble metals are used also a fluorescence signal enhancer layer. A polymeric outer layer may be deposited analogous to the above description using seeded growth from a core/layer particle. A layer such as Au or Ag may also be grown as the outer layer through seeded growth either by reducing gold or silver salts in the presence of a reducing agent, or by first adsorbing small Au/Ag NPs onto the particle to be encapsulated, and then using these Au/Ag NPs as seeds for further growth of the metallic layer. To facilitate the absorption of Au/Ag NPs, or for facilitating seeded growth of the metallic layer directly from the surface of the particle to be encapsulated, facilitating molecules analogous to above can be used. These can be either monomers such as thiols or amino groups directly attached to the surface of the particle or polymers adsorbed to the particle surface.

In an embodiment of the third aspect of the invention, a multilayer particle is synthesized in where the purpose of the layers is to physically separate the different types of fluorophores from each other by introducing a distance between them. To achieve this, a core nanoparticle containing a first fluorophore is synthesized. After purification and isolation, this particle can be used for seeded growth analogous to above to form a first layer containing a second fluorophore in the matrix. Similarly, a second layer can be grown in a next step with a third fluorophore incorporated. It should be noted that each layer is not limited to incorporating only one fluorophore at a time.

In an embodiment, a layered particle is synthesized using an emulsion-based approach by first providing a core particle. Such core particle can act as a seed for the growth of a first layer, or it can be encapsulated by the growth of a matrix inside an emulsion drop. The core particle can be synthesized separately or be used as purchased, such as a quantum dot (core or core/shell semiconductor particle with one or multiple shells) or a fluorescent silica particle. Using an emulsion method for synthesis of a layer, a single particle acting as the core (e.g., quantum dot) is provided to the emulsion droplet in the presence of a surfactant. The core/layer particles are then produced by two main steps. The first step is an aqueous phase (in case of water-in-oil emulsion) encapsulating the core particle with surfactants to form a stable micro emulsion droplet with an organic continuous phase. The second step is for the monomer (such as an alkoxysilane or an organic monomer) to pass the oil-water interface by diffusion and start to polymerize upon catalysis. The thickness of the layer can be optimized by the monomer feed ratio. The quality of core/layer particle can be controlled by reaction conditions including concentration of reactants, temperature, time and stirring rate.

In another embodiment, the core-layer particle can comprise one or more fluorophores in one or multiple layers of polymer. The polymer layer may be composed of one type of polymer or different blended or co-polymers of desired physicochemical properties. This polymer layer can grow from an initiated core surface by controlled radical polymerization such as atom transfer radical polymerization (ATRP) or reversible addition fragmentation chain transfer polymerization (RAFT). Also, the polymer layer can be made of amphiphilic polymer containing hydrophobic and hydrophilic segments. The amphiphilic character of the layer will enable the loading of different fluorophores with different hydrophilicity nature resulting multifluorescent layer. Furthermore, for the polymer layer, different and controlled molecular weights polymers can be obtained by ATRP or RAFT which will be useful when loading small or large fluorophores and/or fluorescent particle (e.g., layer-by-layer deposition).

### Methods of Using the Nanoparticles

According to a fifth aspect of the invention there is provided a method for multiplexed detection of a plurality of target biomolecules using optical encoding using a plurality of nanoparticles according to the first, second or third aspect of the invention. This method is referred to herein as the method of the invention.

The term “optical encoding” is to be interpreted as the nanoparticle having a unique signal by the incorporation of a defined ratio of first and second fluorophores in the nanoparticles. For example, the unique signal may be achieved by incorporating predetermined amounts of the first and second fluorophores, thereby arriving at a nanoparticle type that exhibits a specific emission wavelength and intensity.

The term “nanoparticle type” is to be interpreted as a nanoparticle having a coating that provides binding affinity to a specific detection target and a unique optical encoding. A plurality of (e.g. at least two) nanoparticle types may be used in accordance with the method of the third aspect of the invention, the various types having binding affinity for different targets. Each nanoparticle type will be represented by a plurality of nanoparticles having the same coating and optical encoding.

In an embodiment, each target biomolecule has at least one detection target, and the method comprises the steps of:
a) providing a plurality of nanoparticle types comprising a plurality of nanoparticles according to the first, second or third aspect of the invention, each nanoparticle having a coating that provides binding affinity of the nanoparticle to a type-specific detection target, wherein the combination of the first and second fluorophores in the nanoparticles of each nanoparticle type generate a signal which is unique for that nanoparticle type;
b) providing a sample comprising a plurality of target biomolecules;
c) contacting the sample with the plurality of nanoparticle types, thereby allowing the nanoparticles to bind with the detection targets of the target biomolecules; and
d) optically decoding the fluorophore signals emitted by the nanoparticle of the nanoparticle types bound to the detection target of the target biomolecules by measuring the wavelength and intensity of the emitted signals, thereby detecting the presence and identity of the target biomolecules.

Hereby, a method of simultaneously detecting a significant plurality (e.g. 50-100) of detection targets in situ using optically encoded nanoparticles in a one-step direct manner is provided. This is achieved by tailoring the nanoparticles of each nanoparticle type to achieve distinct and precise incorporation of the first and second fluorophores in well-defined nanoparticles and thereby optically encoding such as outlined above in respect of the first, second and third aspect of the invention.

The term “binding” with a target biomolecule/type specific detection target, a nanoparticle and/or a detection target is to be interpreted as including the alternative of “hybridization” of nucleic acid molecules to each other.

Popular in situ hybridisation applications include spatial biology, spatial mapping, spatial transcriptomics, spatial proteomics and cell mapping.

In an embodiment, the nanoparticle types are in suspension, that is to say the nanoparticles of the nanoparticle types are in suspension.

The detection target may be designed such that the plurality of nanoparticles can attach within at least one optically resolvable pixel forming a cluster of nanoparticles in the spot of detection target, such that the signal intensity becomes higher in this cluster compared to the signal from single nanoparticles that may bind non-specifically to the surrounding, allowing for a localized signal to be detected upon target binding, where the unique identity of the nanoparticle encodes for the target identity. Typically this may be done by performing target amplification prior to decoding, such as rolling-circle amplification (RCA) or other means of localized/clonal amplification (hairpin chain reaction etc.), or by designing probes in a manner similar to traditional fluorescent in situ hybridization (FISH) techniques such that they can form a localized cluster.

The improved fluorophore-nanoparticle complex in particular enables a higher degree of intensity-based multiplexing by allowing for the core to act as an internal reference. In particular, this is important when multiple particles are bound to a detection target in a localized cluster, because in order for intensity-based multiplexing to be fully utilized it is necessary to know how many particles are present in the cluster. Therefore, by having an internal reference, preferably a semiconductor in the core, a higher degree of multiplexing is enabled.

The method allows for nanoparticles with a certain unique identity to bind to a detection target or biomolecule target (DNA/RNA/protein) in situ, where the size of the nanoparticles allows for the penetration of cells and that the nanoparticles can immobilize to the biomolecule target while the excess is washed away. Alternatively, the biomolecule targets are not in cells but are instead immobilized in a 2D or 3D matrix of material, and are still considered to be in situ. For example, the target biomolecules can be immobilized onto the surface of a microscope slide, or in a 3D polymer network, or within tissue where furthermore optionally most of the tissue components except the biomolecule targets are removed. The target biomolecule may be designed such that a plurality of nanoparticles can attach within at least one optically resolvable pixel forming a cluster of nanoparticles in the spot of detection target or target biomolecule comprising detection targets, such that the signal intensity becomes higher in this cluster compared to the signal from single nanoparticles that may bind non-specifically to the surrounding, allowing for a localized signal to be detected upon target binding, and where the unique identity of the nanoparticles that encodes for the target identity can be read/decoded in an isolated manner because the plurality of nanoparticles that bind to one biomolecule target are of the same nanoparticle type. Such signal would therefore be spatially resolved, because each signal identity encoding for the target identity can be read/decoded in an isolated manner with respect to the nanoparticle type, without a significant interference of another nanoparticle type signal within this spatially resolved spot or volume, meaning that even if there may be a small interference by non-specifically bound nanoparticles from another nanoparticle type in the localized spot of signal, the signal from the specifically bound nanoparticles from the correct nanoparticle type is strong enough to decode the identity with confidence. Typically this may be done by performing target amplification prior to decoding, such as rolling-circle amplification (RCA) or other means of localized/clonal amplification (hairpin chain reaction etc.), or by designing probes in a manner similar to traditional fluorescent in situ hybridization (FISH) techniques such that they can form a localized cluster that is spatially resolved, enlarging the size of the original biomolecule target and thereby allowing multiple nanoparticles of the same nanoparticle type to bind to the biomolecule target. Such amplifications methods are sometimes also commonly referred to as clonal amplification methods.

That is to say, the method allows for nanoparticles with a certain unique identity to bind to a detection target or biomolecule target in a sample (DNA/RNA/protein), where the nanoparticles can immobilize onto the detection target allowing for excess non-bound nanoparticles to be washed away from the sample after the contacting step.

In an embodiment the method of the invention may be performed in-situ or ex-situ. The term in-situ refers to the sample contacting the nanoparticles after being immobilized on a 2D or 3D matrix of material. The term ex-situ refers to sample contacting the nanoparticles in an homogeneous solution.

As used herein, the term “2D or 3D matrix of material” refers to a solid phase made of glass, or plastics, such as polystyrene, polyethylene, polymethylmethacrylate, cyclic olefin copolymer, agarose, biodegradable polymers (such as polylactide, poly(glycolides), poly(ε-caprolactone)) or combinations thereof. The solid phase may be coated with biomolecules such as proteins and/or nucleic acids to improve the immobilization of the sample and/or passivation of the surface. Alternatively the solid phase may not be coated.

As used herein, the term “sample” refers to any sample containing a target biomolecule (e.g. a plurality of target biomolecules). For example, a sample may be a biological sample, such as a body fluid sample, a tissue sample or a single cell. The sample may further comprise a mixture of biomolecules including proteins and/or nucleic acids in an homogeneous solution or immobilised on a “2D” or “3D” matrix of material via affinity, electrostatic, covalent, or Van der Waals interactions, or combinations thereof.

As used herein, the term “detection target” refers to any target in the sample to which the nanoparticle(s) can bind.

In an embodiment the detection target is a polynucleotide sequence. By the term “polynucleotide sequence” we include any biopolymer composed of nucleotide monomers in a chain, for example DNA and/or cDNA and/or RNA and/or a protein.

In another embodiment, the detection target is designed such that a plurality of nanoparticles can attach within at least one optically resolvable pixel forming a cluster of nanoparticles in the spot of detection target, such that the signal intensity becomes higher in this cluster compared to the signal from single nanoparticles that may bind non-specifically to the surrounding, allowing for a localized signal to be detected upon target binding, where the unique identity of the nanoparticle encodes for the target identity.

Typically this may be done by performing target amplification prior to decoding, such as rolling-circle amplification (RCA), Polymerase Chain Reaction (PCR), Reverse Transcriptase Polymerase Chain Reaction (RT-PCR), Loop mediated isothermal amplification (LAMP) or other means of localized/clonal amplification (hairpin chain reaction etc.), or by designing probes in a manner similar to traditional fluorescent in situ hybridization (FISH) techniques such that they can form a localized cluster. These methods may be based on the use of exonuclease, endonuclease, transposase or CRISPR/Cas-based enzymatic activity prior to amplification. The amplification procedure may also comprise a combination of two or more of the techniques above.

By using optically encoded nanoparticles according to the first, second or third aspect of the invention, i.e. nanoparticles that comprise precisely controlled ratios of the first and second fluorophores such that each the combination of ratios represents a unique identity, this enables a higher number of multiple identities compared to single colors alone. The number of combinations becomes n^{m}−1, where n is the number of intensity levels and m the number of colors.

To improve the accessibility of the nanoparticles to the target biomolecules in tissue samples and/or single cells, the latter can be locally permeabilized to allow the target biomolecules to locally diffuse to a 2D or 3D solid phase as defined herein, where they are immobilized via physisorption, electrostatic, hybridization or affinity interactions. In this way, the target biomolecules are fully accessible without the tissue and/or cell matrix while spatial information can still be retrieved.

According to some embodiments, before step c the method of the invention may comprise a step of preparing the target biomolecules for binding with the nanoparticles, such as binding the target biomolecules with at least one molecule comprising the at least one detection target, and/or amplifying the detection targets in situ. Optionally in this step, the target biomolecule is prepared by binding to it at least one molecule such as a barcoded nucleic acid, padlock probe or initiator sequence for subsequent amplification.

This enables the NP probes to be used in assays where enzymatic amplification is omitted, such as regular in situ hybridization (ISH) methods where at least one, but preferably multiple detection targets are bound to a biomolecule in order to generate a stronger signal by binding multiple NPs to a biomolecule.

According to some embodiments, the detection target comprises a nucleic acid molecule, which is, or facilitates a molecule that is, amplified using RCA or multiple hybridization events.

Hence, the signal detection becomes easier and more robust due to (i) a higher signal intensity (ii) the necessity of multiple binding events to generate said signal to avoid randomly immobilized nanoparticles and (iii) utilizing the specificity of molecular tools such as padlock probes for the amplification of the target, avoiding non-targets to be amplified and therefore detected. Similarly, other amplification methods utilizing the binding of for example two or more detection targets in close proximity to allow subsequent hybridizations to generate a stronger but specific signal can be achieved.

According to some embodiments, the decoding is effected by optical decoding such as by optical imaging/fluorescent imaging.

Alternatively, the decoding can be performed in a flow-cytometry-type apparatus that allows the sample to flow through a narrow nozzle with dimensions comparable to a discrete element presenting a specific target biomolecule. The “discrete element” as described herein can be a single cell or a microparticle. The said microparticle can have a probe according to the definition herein to specifically immobilize the target molecule upon presentation to the coded nanoparticles.

This enables high resolution spatial information to be collected with the throughput and resolution of the imaging system and can enable large areas to be scanned.

In another embodiment, the target biomolecule is further co-labelled with small molecule dyes/probes together with the nanoparticles.

In an embodiment, the method comprises the step of further providing one or more molecular probes, each molecular probe comprises a fluorophore that is bound to a nucleic acid molecule, an antigen or an antibody providing binding affinity of the molecular probe to the specific detection target.

As used herein, the term “molecular probe(s)” is referred to interchangeably as “detection oligo(s)”.

The fluorophore of the molecular probe may be any fluorophore, such as any fluorophore detailed herein.

This further improves both robustness and multiplexing where signal detection will rely on both nanoparticle binding event as well as molecular probe binding events to ensure that non-specifically bound nanoparticles are filtered from data analysis. For example, co-localization analysis of nanoprobe signal and detection oligo signal, allowing the removal of false positives from either nanoprobe signal only and detection oligo signal only. This is useful both in-situ and ex-situ, both in tissue and outside tissue. In addition, this can further be utilized to increase the multiplexing capacity by introducing different color of fluorophores acting as molecular probes.

For the avoidance of doubt, multiplexing will be achieved as long as there are at least two nanoparticle types used. For example, multiplexing may be achieved with at least 3, 4, 5, 6, 7, 8, 9 or 10 nanoparticle types

Particular advantages and unexpected effects of the method of the invention include:
Time can be saved, experimentally and imaging wise, due to one step vs. multiple for sequencing;
Costs can be saved due to fewer steps;
The method is less sensitive to handling errors such as sample integrity and other risks of failure during sequencing procedures;
The nanoparticles shelf-life (emission and coating stability) was shown to unexpectedly long with a period of over 1 year which makes the kits particularly suitable for diagnostics applications as the outcome of the tests become less sensitive to storage and handling errors;
The method allows for an easier data analysis due to one set of images instead of multiple which requires aligning;
The method can be used with simple hardware setup by a regularly trained lab person;
The method does not require fluidic solutions for iterative chemistry/imaging steps;
The method may lower the barrier for adoption of in-situ methods in regular laboratories that lack advanced facilities and instrumentation; and
The present invention may be used for multiple applications where a localized signal needs detection/identification.

### Kit-of-Parts

According to a sixth aspect of the invention, there is provided a kit-of-parts, comprising, in separate containers:
(i) a plurality of nanoparticles according to the first, second or third aspect of the invention;
(ii) a probing buffer, comprising a solution with controlled pH, salt concentration and additives facilitating specific detection target binding of the nanoparticle(s); and
(iii) instructions for use of the kit in the method according to the fifth aspect of the invention.

In an embodiment, the kit comprises a plurality of nanoparticle types each in separate containers and each comprising a plurality of nanoparticles according to the first, second or third aspect of the invention, wherein the combination of the first and second fluorophores in the nanoparticles of each nanoparticle type generate a signal which is unique for that nanoparticle type.

In a further embodiment, the nanoparticles of each nanoparticle type comprise a coating as defined in the first, second or third aspect of the invention.

In another embodiment, the nanoparticles of each nanoparticle type comprise a coating that provides binding affinity of the nanoparticle to a specific detection target.

In a further embodiment, the nanoparticles of each nanoparticle comprise a coating that comprises a plurality of linkers having a conjugating group, such as a conjugating group as defined above in the first, second or third aspect of the invention. This provides for a kit containing multiple nanoparticle types that have a coating that allows for rapid and custom modification of the nanoparticles with various targets to easily create new panels as wished for by the end user.

For example, the linker may by conjugated to a detection probe as defined herein, optionally wherein the detection probe is an antigen or an antibody.

Therefore, in an embodiment the kit-of-part comprises ingredients for providing the one or more nanoparticle type(s) with binding affinity to a specific detection target, comprising a reaction buffer facilitating the binding of detection probe to the linker, a washing buffer, and a suspension buffer to suspend the nanoparticles in after the introduction of the binding affinity to the coating.

For the avoidance of doubt, the nanoparticles of the nanoparticle type(s) of the fourth, fifth and sixth aspect of the invention may comprise any of the features of the nanoparticles of the first, second and/or third aspect of the invention.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure. Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

Wherever the word ‘about’ is employed herein in the context of amounts, for example absolute amounts, such as weights, volumes, sizes, diameters, etc., or relative amounts (e.g. percentages) of individual constituents in a composition or a component of a composition (including concentrations and ratios), timeframes, and parameters such as temperatures, pressure, etc., it will be appreciated that such variables are approximate and as such may vary by ±10%, for example ±5% and preferably ±2% (e.g. ±1%) from the actual numbers specified herein. This is the case even if such numbers are presented as percentages in the first place (for example ‘about 10%’ may mean±10% about the number 10, which is anything between 9% and 11%).

For the avoidance of doubt, unless otherwise specified it is intended that any embodiments described above may be couple with the embodiments of the detailed description below if reasonably plausible.

### DESCRIPTION OF THE FIGURES

FIG. 1 A) schematic figure of a nanoparticle according to the invention comprising a semiconductor fluorophore core and a first layer with a second fluorophore; B) schematic figure of a nanoparticle according to the invention comprising a cluster of semiconductor fluorophore acting as a core and a first layer with a second fluorophore; C) a schematic figure of a nanoparticle according to the invention comprising a core having a first fluorophore, a first layer with a second fluorophore and an outer layer composed of a metal such as gold or silver; D) a schematic figure of a nanoparticle according to the invention comprising a core having a first fluorophore, a first layer with a second fluorophore and a second layer with a third fluorophore.

Although the nanoparticles in FIG. 1 are depicted as being spherical, it should be realized that this is of course merely a schematic representation, and in fact the nanoparticles may also be irregular in shape. Likewise, the proportions between the nanoparticle diameter and the coating thickness are merely illustrative and these proportions may vary depending on factors such as the desired dissolution time, the numbers of layers in the coating, etc.

FIG. 2 TEM image of a core-layer NP where the core is a CdSe/ZnS quantum dot and the layer is a silica layer containing different ratios of second fluorophore(s). The quantum dot core is ˜5 nm in diameter and the core-layer is ˜60 nm in diameter.

FIG. 3 TEM image of a core-layer NP where the core is comprised of multiple CdSe/ZnS quantum dots and the layer is a silica layer containing different ratios of second fluorophore(s).

FIG. 4 Emission combination plots of nanoparticles according to the invention a) Plot 1 showing Cy3/Cy5 ratio variation in the presence of QD_B; b) Plot 2: Cy3/Cy5 ratio variation in the presence of QD_C; and c) Plot 3: Cy3/Cy5 ratio variation in the presence of QD_B+QD_C simultaneously.

FIG. 5 SEM images of core NPs with a first fluorophore (Cy3) with 60 nm size in diameter and core-layer NPs where a layer is grown around the core containing a second fluorophore (Cy5) to yield approximately 100 nm in diameter particles.

FIG. 6 Relative fluorescence emission from core NPs (60 nm) and core-layer NPs (100 nm), where the core contains a first fluorophore (Cy3) and layer 1 contains a second fluorophore (Cy5).

FIG. 7 SEM image of NPs with a outer layer of Au.

FIG. 8 Schematic showing the principle of the method of the invention, wherein (a) shows the situation where one or more nanoparticles (NP) has binding affinity to bind to a specific detection target in a target biomolecule (rolling circle product (RCP)), and (b) shows the situation where the nanoparticle lack binding affinity to the target biomolecule (i.e. no detection target specific for the nanoparticle is present in the biomolecule).

FIG. 9 Graphic showing the principles for the coating of the nanoparticles in accordance with the method of the invention.

FIG. 10 Signal emission analysis from 3500 spots analyzed using the method of binding multiple NPs to the biomolecule target via multiple detection targets co-labelled with two different molecular probes (detection oligos, Atto425 and AF750).

FIG. 11 14-plex readout of the 3500 spots from signal emissions in FIG. 10 showing how the optical encoding can be used to achieve a high plex biomolecule counting and identification, including filtering out spots that either could not be classified or were not co-localized with the molecular probe.

FIG. 12 Fluorescent image of rolling-circle amplified biomolecules immobilized on a surface probed with 7 different nanoparticle types and 2 different molecular probes (detection oligos) which yield the data in FIG. 10 and FIG. 11.

FIG. 13 Signal emission analysis of Cy3 & Cy5 of 3-color (Cy3/Cy5/A425) encoded NP library where A425 emission is set to 0.

FIG. 14 Size of NP batches before and after a first layer formation measured by DLS.

FIG. 15 Signal emission analysis of Cy3 & Cy5 of 3-color (Cy3/Cy5/A425) encoded NP library where A425 emission is set to 16.

FIG. 16 Signal emission analysis of A425 & Cy3 of 3-color (Cy3/Cy5/A425) encoded NP library where A425 emission is set to 16.

FIG. 17 Signal emission analysis of A425 & Cy5 of 3-color (Cy3/Cy5/A425) encoded NP library where A425 emission is set to 16.

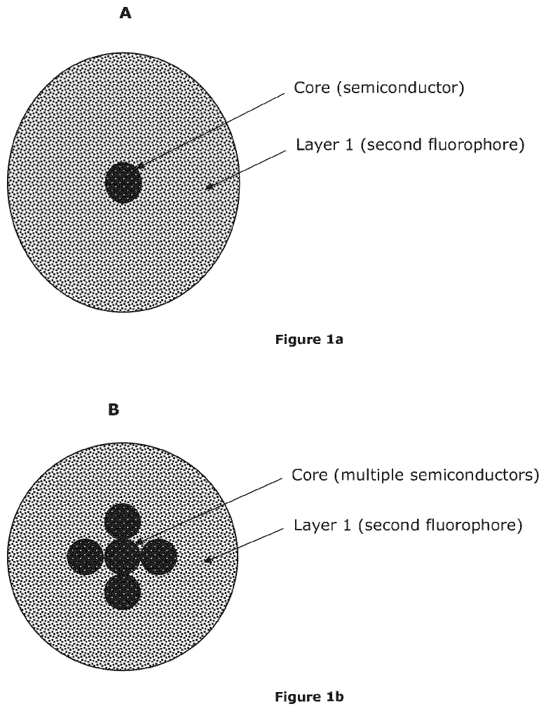

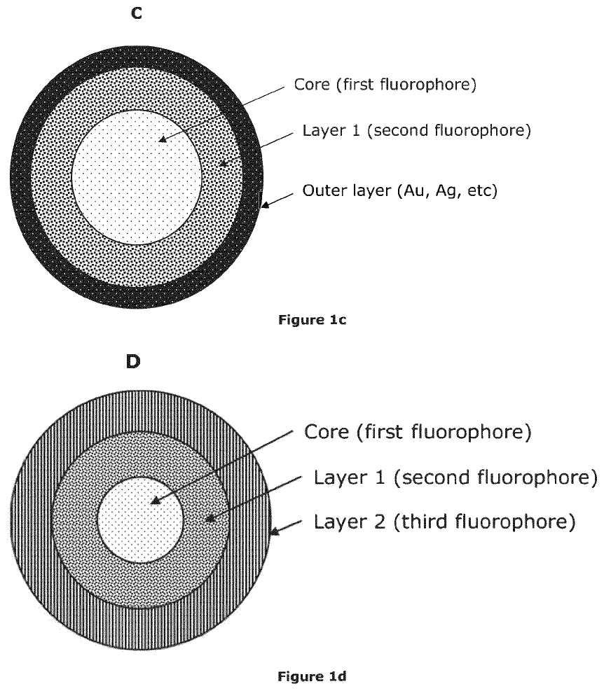

The person skilled in the art realizes that the present disclosure is not limited to the preferred embodiments described above. The person skilled in the art further realizes that modifications and variations are possible within the scope of the appended claims. Additionally, variations to the disclosed embodiments can be understood and effected by the skilled person in practicing the claimed disclosure, from a study of the drawings, the disclosure, and the appended claims.

### EXAMPLES

### Example 1

### Materials

Cyanine 3 NHS ester (Cy3-NHS), Cyanine 5 NHS ester (Cy5-NHS) were purchased from Lumiprobe GmbH, Germany. Dimethyl Sulfoxide (DMSO) (≥99.9%), Tetrahydrofuran (THF (99%) (3-aminopropyl) triethoxysilane (APTES), ammonium hydroxide (NH₄OH) (28% NH₃ in H₂O, ≥99.99%), tetraethyl orthosilicate (TEOS) (99.999%), 11-azidoundecyltriethoxysilane (97%), ADIBO-PEG4-acid (90%), Polyvinylpyrrolidone (PVP) (Mw 10 kDa), Triton X-100 (polyethylene glycol tert-octylphenyl ether, n=9-10), CdSe/ZnS core-layer QDs (5 mg mL⁻¹, λₑₘ 520 nm) in toluene (QD_A) and CdSe/ZnS core-layer QDs (1 mg mL⁻¹, λₑₘ 580 nm) in H2O (QD_B) were purchased from Sigma Aldrich, Sweden. Qdot™ 655 Streptavidin Conjugate (QD_C) (1 μM in H₂O) was purchased from ThemoFisher Scientific, Sweden. Ethanol (EtOH) absolute (99.8%) was purchased from VWR, Sweden. Ultra-pure MilliQ water used from MilliQ (IQ 7010) system.

### Fluorophore Stock

Stock solutions of fluorophores were prepared by adding 1 ml of DMSO to 1 mg of respective fluorophore.

### Example Nanoparticle Synthetic Method 1

Encoded NPs were synthesized by adding either of [0, 0.8, 1.6, 3.2, 6.4] μl Cy3-NHS and/or Cy5-NHS and/or Cy7-NHS respectively to 4 μL of APTES solution and raising the temperature to 37° C. during stirring or shaking. The APTES solution was prepared by first adding 10 μl of APTES to 990 μl of EtOH. For example, to produce a nanoparticle type batch of encoding (8:16), 0.8 μL of Cy3-NHS and 1.6 μL of Cy5-NHS was added.

Furthermore, to produce a core-layer nanoparticle type batch with a QD in the core and 2 fluorophores in a first layer, of encoding (8:16:QD_B), 0.8 μL of Cy3-NHS and 1.6 μL of Cy5-NHS was added to the mixture containing QD_B during below layer formation step.

For the core-layer synthesis, a solution of PVP (Mw 10 kDa) in EtOH was prepared at a concentration of 5 wt. %. Aqueous QDs (QD_B, QD_C or QD_D, 0.01 μM) were mixed with PVP solution (870 μL) at RT for overnight. To initiate the layer formation, this solution was added to above fluorophore-APTES solution followed by the addition of H₂O (40 μL) and NH₄OH (28%, 56 μL). TEOS (40 μL) was added under vigorous shaking. The reaction was further agitated at RT for overnight. The resultant suspension was isolated by centrifugation.

The precipitated particles were redispersed and washed in EtOH for 3 times. Finally, the core-layer NPs was redispersed in 1 mL EtOH.

### Example Nanoparticle Synthetic Method 2

For the core-layer synthesis, an aqueous solution of QDs (QD_B, or QD_C, 0.01 μM) were mixed with a solution of Triton X-100 (1.9 g), cyclohexane (7.5 mL) and 1-hexanol (1.8 mL). The mixture was stirred for 15 min at RT followed by addition of the above fluorophore-APTES solution under stirring. Ammonium hydroxide (25%, 240 μL) was added followed by TEOS (100 μL) to the previous mixture under vigorous stirring. The mixture was stirred for 73 h at RT. The resultant suspension was recovered by adding 10 mL acetone followed by centrifugation at 5550 RCF for 10 min to separate the NPs. The core-layer NPs were washed in sequence with 24 mL 1-butanol, 24 mL isopropanol, and 24 mL EtOH. Finally, the core-layer NPs were redispersed in 10 mL EtOH.

### Example Nanoparticle Synthetic Method 3

For the core-layer synthesis, an organic solution of QDs (QD_A, 0.05 mg/mL) was mixed with a solution of Triton X-100 (0.945 g), cyclohexane (5 mL) and 1-hexanol (0.9 mL). The mixture was stirred for 15 min at RT followed by addition of H₂O (0.19 mL) and the above fluorophore-APTES solution under stirring. A successful addition of ammonium hydroxide (28%, 30 μL) followed by TEOS (30 μL) to the previous mixture under vigorous stirring. The mixture was continued to agitate for overnight at RT. Another 170 μL TEOS was added under vigorous stirring and the mixture was further stirred for 24 h at RT. The resultant suspension was recovered by adding 7.5 mL acetone followed by centrifugation at 5550 RCF for 10 min to separate the NPs. The core-layer NPs were washed successively with 20 mL 1-butanol/hexane (1/1 v/v), 20 mL isopropanol/hexane (1/1 v/v), 20 mL EtOH/hexane (1/1 v/v), and 20 mL EtOH. The core-layer NPs were dispersed in 10 mL H₂O, and further washed with 10 mL methanol. Finally, the core-layer NPs were redispersed in 5 mL methanol.

In one embodiment, a 15-plex encoded NP system was synthesized using above method with the following encodings: (8:0:QD_B, 16:0:QD_B, 32:0:QD_B, 0:8:QD_B. 8:8:QD_B, 16:8:QD_B, 32:8:QD_B, 0:16:QD_B, 8:16:QD_B, 16:16:QD_B, 32:16:QD_B, 0:32:QD_B, 8:32:QD_B, 16:32:QD_B, 32:32:QD_B).

In another embodiment, a 30-plex encoded NP system was synthesized using above method with the following encodings or a sub-set of these: (8:0:QD_B, 16:0:QD_B, 32:0:QD_B, 0:8:QD_B. 8:8:QD_B, 16:8:QD_B, 32:8:QD_B, 0:16:QD_B, 8:16:QD_B, 16:16:QD_B, 32:16:QD_B, 0:32:QD_B, 8:32:QD_B, 16:32:QD_B, 32:32:QD_B) and (8:0:QD_C, 16:0:QD_C, 32:0:QD_C, 0:8:QD_C. 8:8:QD_C, 16:8:QD_C, 32:8:QD_C, 0:16:QD_C, 8:16:QD_C, 16:16:QD_C, 32:16:QD_C, 0:32:QD_C, 8:32:QD_C, 16:32:QD_C, 32:32:QD_C).

### Example Nanoparticle Synthetic Method 4

Core-layer NPs were synthesized by first synthesizing fluorescent core NPs. Core NPs were synthesized by adding 6.4 μl Cy3-NHS to 4 μL of APTES solution and raising the temperature to 37° C. during stirring or shaking. The APTES solution was prepared by first adding 10 μl of APTES to 990 μl of EtOH. After 10 minutes, 29 μl of H₂O was added followed by 888 μL EtOH and the temperature was raised to 55° C. Finally, 41 μl of NH₄OH was added followed by 28 μl of TEOS during vigorous stirring. After 2 hours, the NPs were washed by centrifugation 3 times using EtOH. The NPs were then redispersed in 100 μL EtOH with an average diameter of approximately 40-60 nm. For the synthesis of an additional layer, first 6.4 μl Cy5-NHS was added to 4 μL of APTES solution and raising the temperature to 37° C. during stirring or shaking. The APTES solution was prepared by first adding 10 μl of APTES to 990 μl of EtOH. After 10 minutes the 100 μL of above core NPs were added to the solution, followed by 29 μl of H₂O and 788 μL EtOH and the temperature was raised to 55° C. Finally, 54 μl of NH₄OH was added followed by 20 μl of TEOS during vigorous stirring. After 6 hours, the NPs were washed by centrifugation 3 times using EtOH. The NPs were then redispersed in 1 mL EtOH with an average size of 100-120 nm. Subsequent layers could be added by repeating above procedure.

Example Nanoparticle synthetic Method 5

The particles prepared by methods 1-4 were further sputtered with a layer of Au by conventional sputtering methods.

Example Nanoparticle synthetic Method 6

The following method is a variation of nanoparticle synthetic method 4.

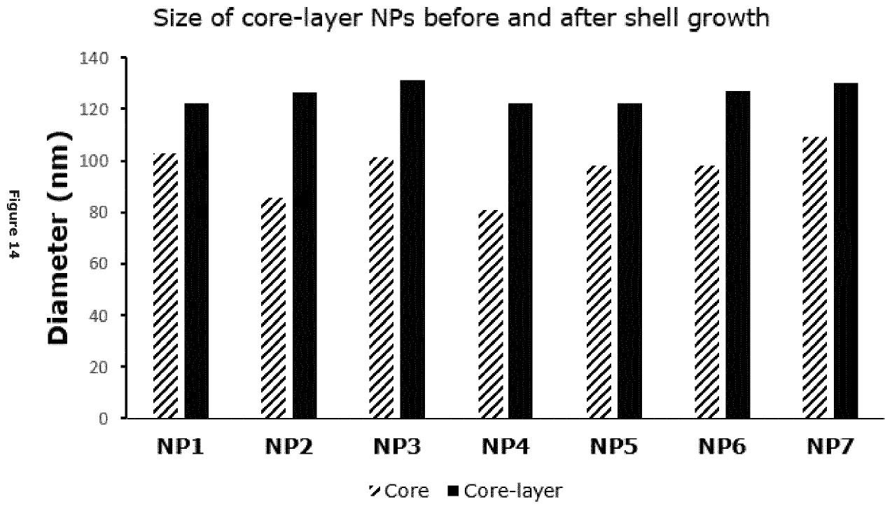

Encoded core-layer NPs were synthesized by first synthesizing fluorescent core NPs. Core NPs were synthesized by adding either of [0, 0.8, 1.6, 3.2, 6.4] μL Cy3-NHS, Cy5-NHS, Cy7-NHS respectively to 4 μL 10% of APTES EtOH solution and raising the temperature to 37° C. during stirring or shaking. After 1 hour of reaction, 888 μL EtOH was added followed by 38 μL of H₂O and the temperature was raised to 55° C. Finally, 54 μL of NH₄OH (28%) was added followed by 38 μL of TEOS during vigorous stirring. After 2 hours of reaction, the NPs were washed by centrifugation 3 times using EtOH and redispersed in 100 μL EtOH with an average diameter of approximately 80-100 nm. For the synthesis of an additional layer, first 1.6 μL Atto425-NHS (A425) was added to 4 μL of 1% APTES solution in EtOH and the temperature raised to 37° C. during stirring or shaking. After 2 hours, the 100 μL of above core NPs were added to the solution, followed by 13 μL of H₂O and 500 μL EtOH and the temperature was raised to 65° C. Finally, 20 μL of NH₄OH was added followed by 24 μL of TEOS during vigorous stirring. After 24 hours of reaction, the NPs were washed by centrifugation 3 times using EtOH. The NPs were then redispersed in 600 uL EtOH with an average size of 120-130 nm (FIG. 14). Subsequent layers could be added by repeating above procedure.

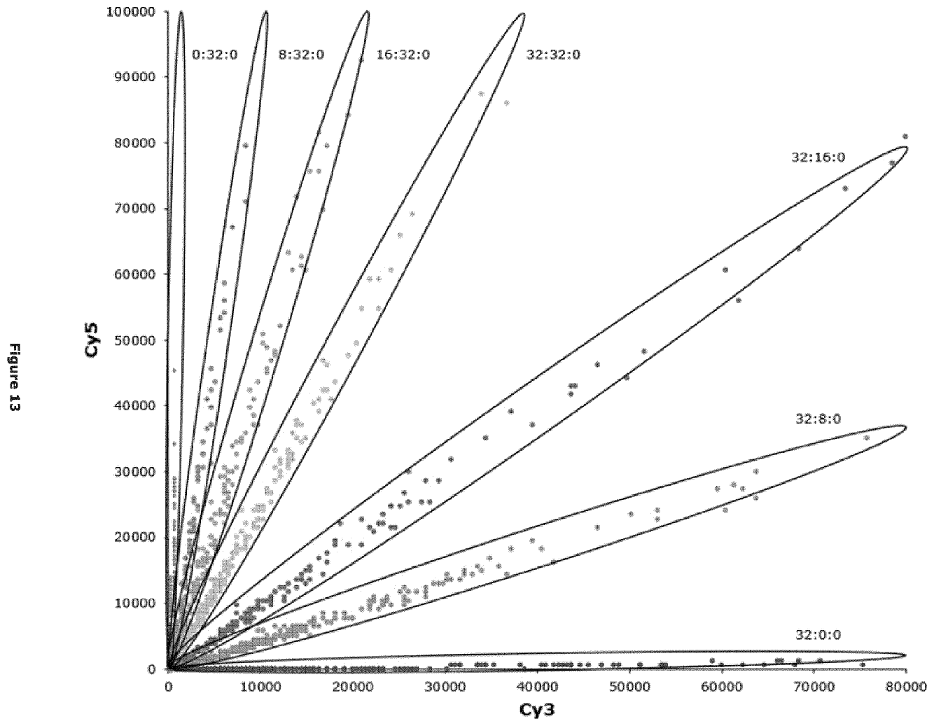

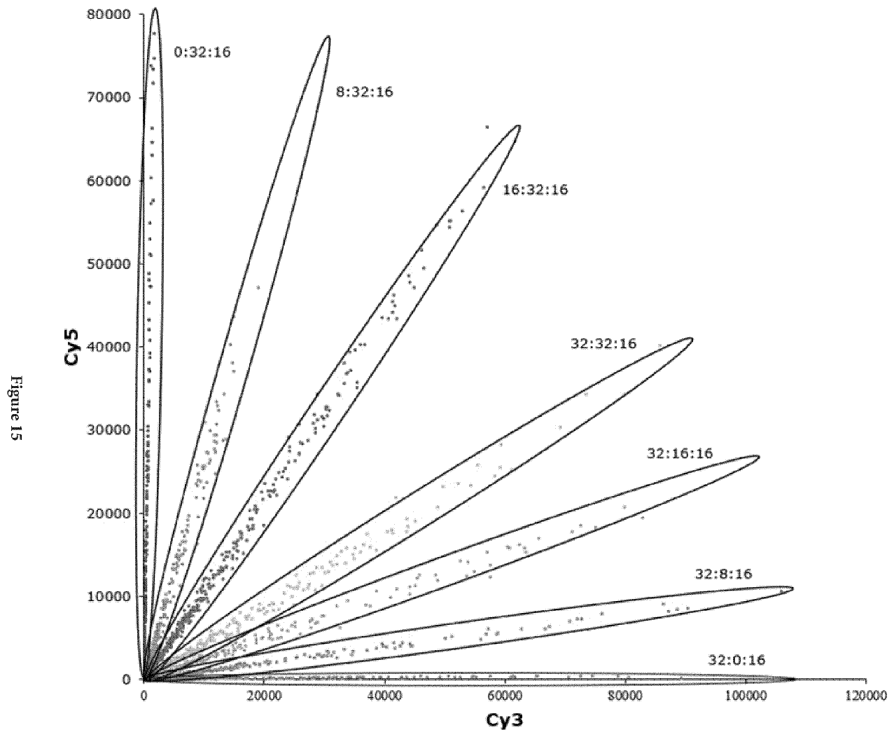

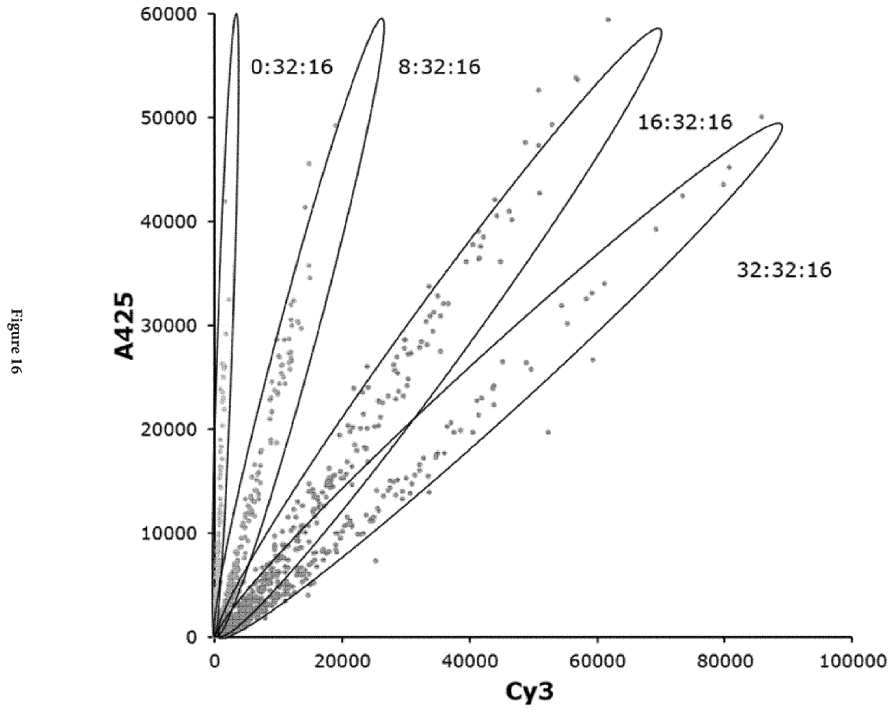

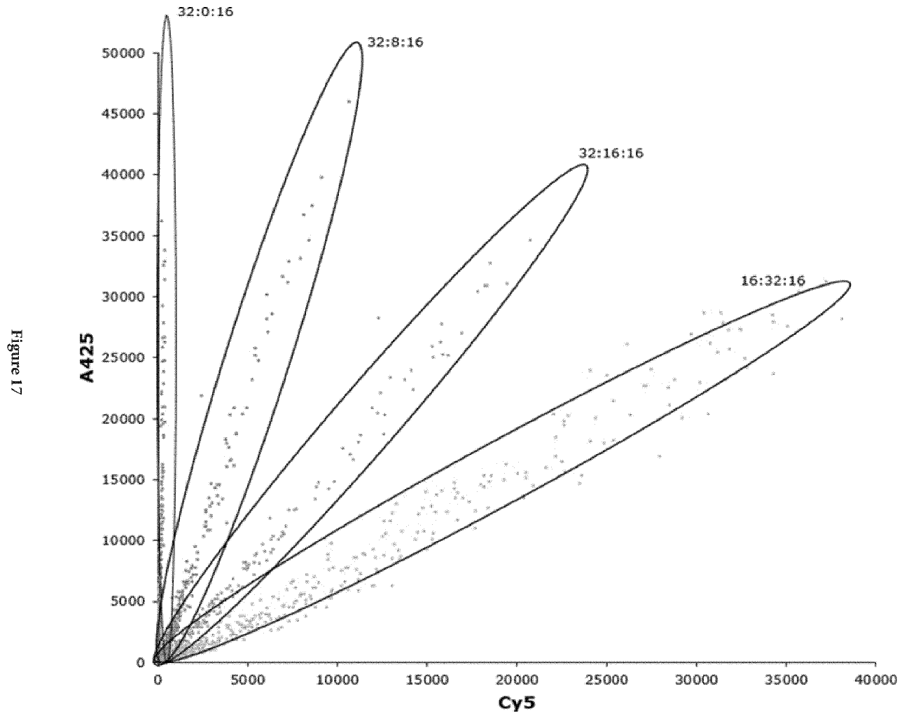

In an embodiment, a 14-plex encoded NP system was synthesized using above method with the following encodings (Cy3/Cy5/Atto425): (0:32:0, 8:32:0, 16:32:0, 32:32:0, 32:16:0.32:8:0, 32:0:0, 0:32:16, 8:32:16, 16:32:16, 32:32:16, 32:16:16. 32:8:16, 32:0:16), and the signal emission analysis of this system can be seen in FIG. 13, where A425 emission is set to 0, and in FIG. 15, FIG. 16 and FIG. 17 where A425 emission is set to 16. The size before and after layer formation around the core NPs was measured by DLS and can be seen in FIG. 14.

This example shows that encoding can be enhanced by adding a layer containing a third color to already encoded core NPs as the emissions from the core-layer NPs in FIG. 15 remain well separated without interference from the added layer. This can be seen by comparing FIG. 15 to the core-only emissions in FIG. 13 which lacks a layer around the core. FIG. 16 and FIG. 17 show the emissions from the core-layer NPs relative to the third color in the layer (A425) and that the emissions are well separated relative to this third color as well, showing both the capability of adding multiple third color levels (in this case two levels, 0 and 16 are used), as well as utilizing the third color as an internal reference to enable more levels to be achieved with the first two colors. This conclusion is drawn by the fact that the ratiometric read-out between all three colors is linear and well separated, which is the requirement for being able to add levels within each color. For example, by adding further levels in the A425 color, the ratios relative to either Cy3 or Cy5 will be different, but because they are linear they can be controlled to be separable in the read-out, thereby achieving a system capable of higher encoding. Likewise, if the strategy of using the third color as an internal reference is used, then it is also a pre-requisite that the ratiometric read-out between Cy3/A425 and Cy5/A425 follows a linear trend and is well separated, meaning that when these intensities are normalized to A425 they can still be used for an encoded read-out. This way, a Cy3/Cy5/A425 encoding of 32:32:16 and 16:16:16 would have the same ratio for Cy3/Cy5, but different Cy3/A425 and Cy5/A425 readouts thereby enabling more levels than previously possible with only two colors.

In another embodiment, a 21-plex encoded NP system was synthesized using above method with the following encodings (Cy3/Cy5/Atto425): (0:32:0, 8:32:0, 16:32:0, 32:32:0, 32:16:0, 32:8:0, 32:0:0, 0:32:16, 8:32:16, 16:32:16, 32:32:16, 32:16:16, 32:8:16, 32:0:16, 0:32:32, 8:32:32, 16:32:32, 32:32:32, 32:16:32, 32:8:32, 32:0:32).

In another embodiment, a 28-plex encoded NP system was synthesized using above method with the following encodings (Cy3/Cy5/Atto425): (0:32:0, 8:32:0, 16:32:0, 32:32:0, 32:16:0, 32:8:0, 32:0:0, 0:32:16, 8:32:16, 16:32:16, 32:32:16, 32:16:16, 32:8:16, 32:0:16, 0:32:32, 8:32:32, 16:32:32, 32:32:32, 32:16:32, 32:8:32, 32:0:32, 0:32:64, 8:32:64, 16:32:64, 32:32:64, 32:16:64, 32:8:64, 32:0:64).

This example shows the high multiplexing capability of the nanoparticles able to be achieved by this process.

### Analysis of Nanoparticles Prepared by any One of Synthetic Methods 1 to 4

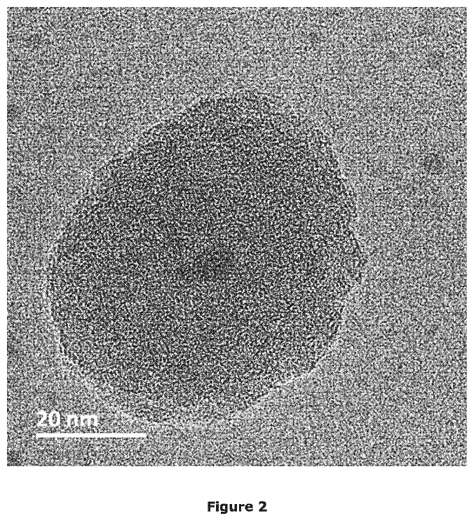

FIG. 2 is TEM image of a core-layer NP prepared according to method 1 where the core is a CdSe/ZnS quantum dot and the layer is a silica layer containing different ratios of second fluorophore(s). The quantum dot core is ˜5 nm in diameter and the core-layer is ˜60 nm in diameter.

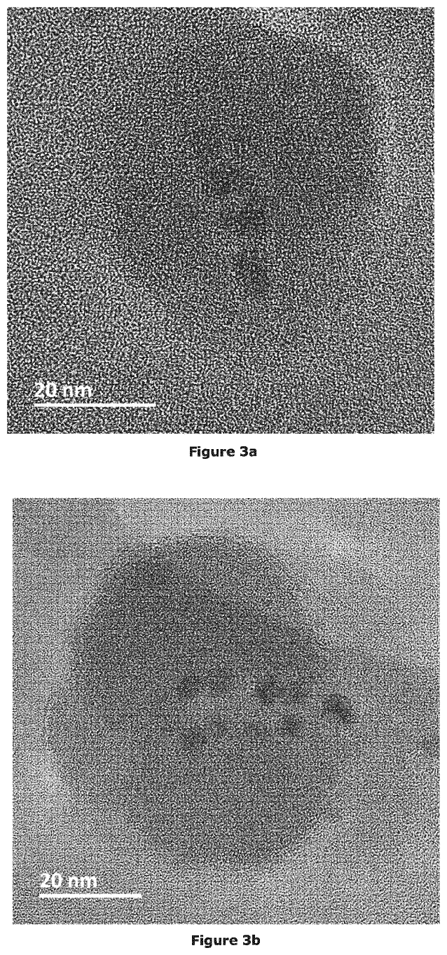

FIG. 3 is TEM image of a core-layer NP prepared according to method 2 where the core is comprised of multiple CdSe/ZnS quantum dots and the layer is a silica layer containing different ratios of second fluorophore(s).

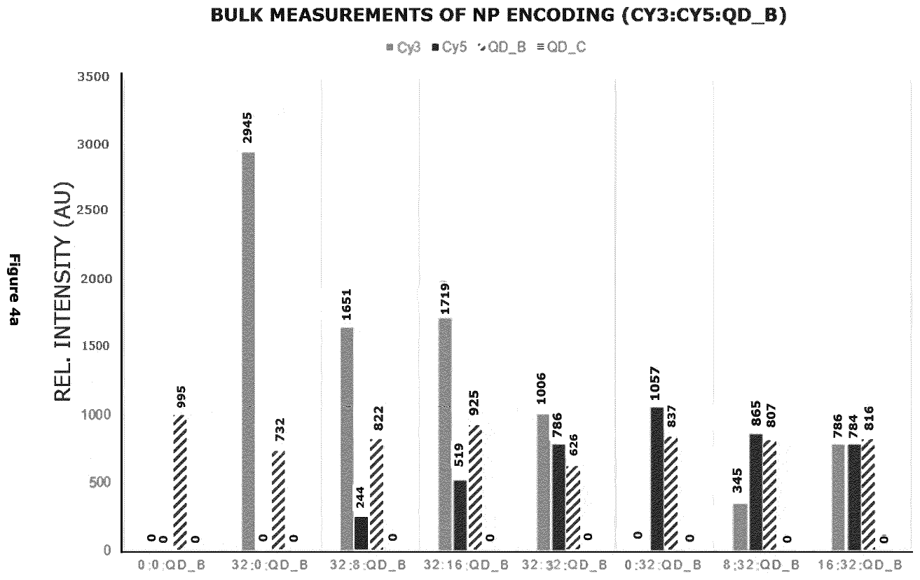

The emission combination plots of nanoparticles according to the invention as prepared according to a sub-set of method 1 are shown in FIG. 4. FIG. 4 a) shows Cy3/Cy5 ratio variation in the presence of QD_B; b) shows Cy3/Cy5 ratio variation in the presence of QD_C; and c) shows Cy3/Cy5 ratio variation in the presence of QD_B+QD_C simultaneously.

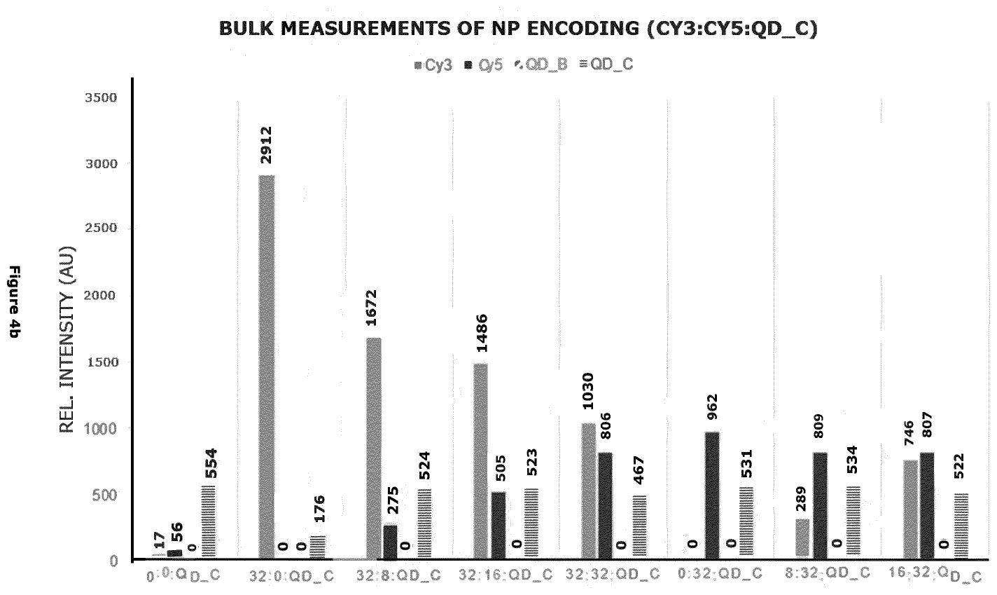

SEM images of core NPs synthesized by method 4 are shown in FIG. 5 with a first fluorophore (Cy3) with 60 nm size in diameter and core-layer NPs where a layer is grown around the core containing a second fluorophore (Cy5) to yield approximately 100 nm in diameter particles.

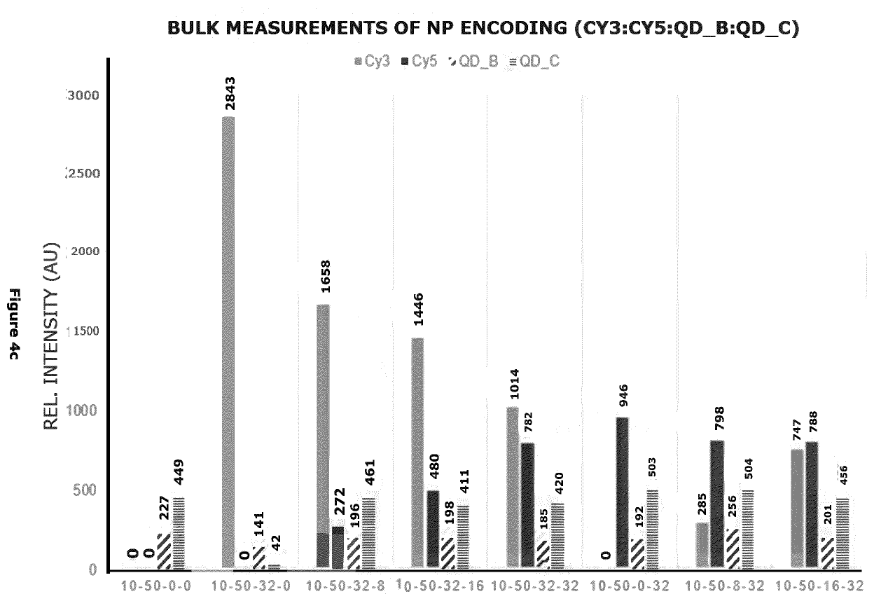

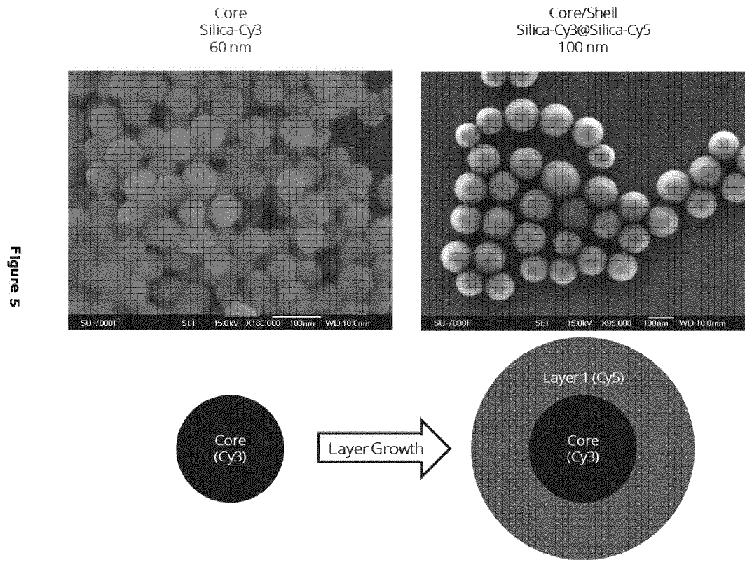

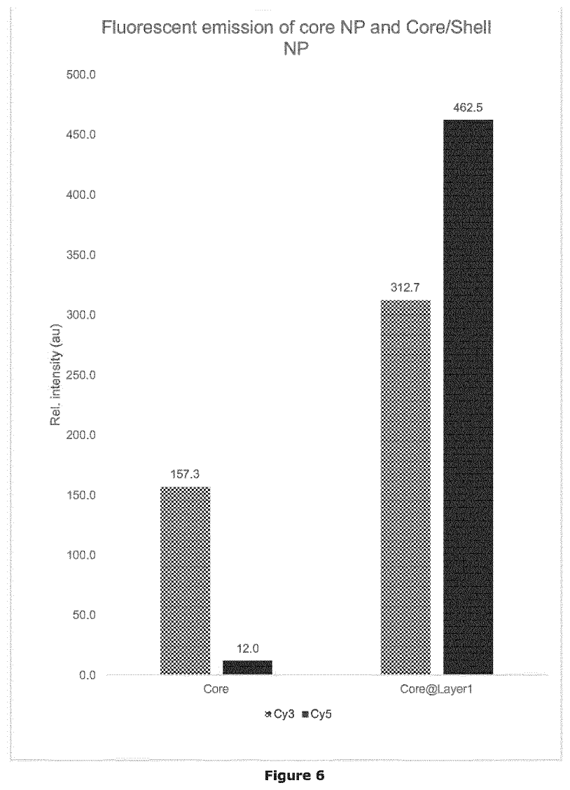

The relative fluorescence emission from core NPs (60 nm) and core-layer NPs (100 nm) synthesized by method 4 can be seen in FIG. 6 where the core contains a first fluorophore (Cy3) and layer 1 contains a second fluorophore (Cy5).

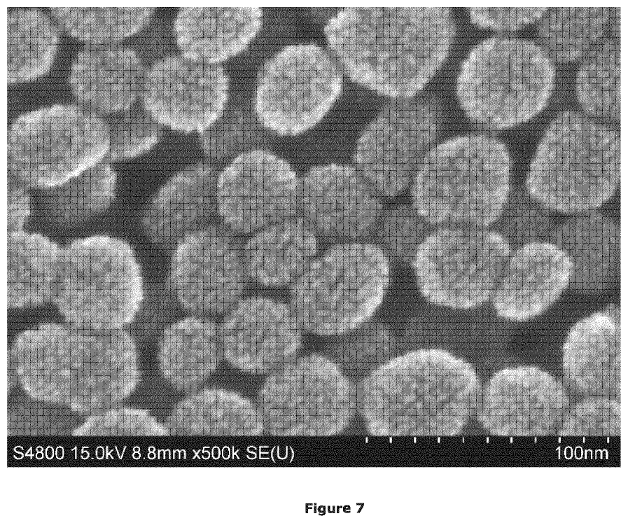

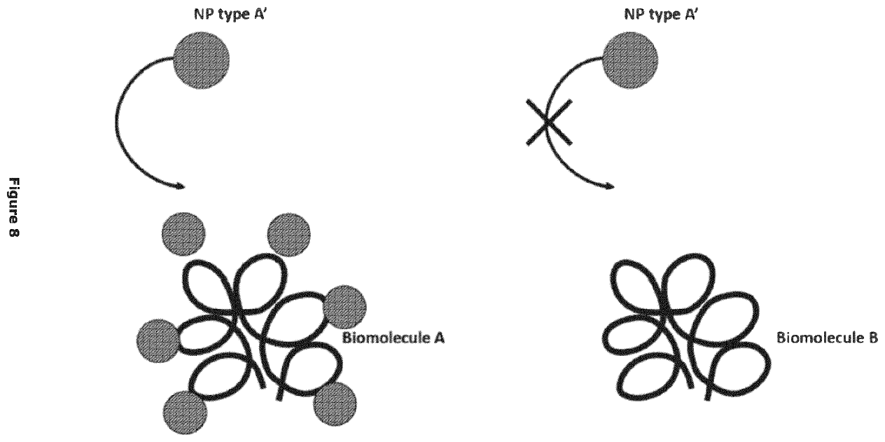

SEM image of NPs with a outer layer of Au as synthesized by method 5 can be seen in FIG. 7.

### Example 2

The following example details the preparation of nanoparticles comprising multiple fluorophores in a silica matrix. The nanoparticles do not comprise a core-layer structure as defined in the claims, but are included herein to demonstrate the principle of multiplexing achieved by the method of the invention.

### Nanoparticle Synthesis

### Materials

Cyanine 3 NHS ester (Cy3-NHS), Cyanine 5 NHS ester (Cy5-NHS) were purchased from Lumiprobe GmbH, Germany. Dimethyl Sulfoxide (DMSO) (≥99.9%), Tetrahydrofuran (THF (99%) (3-aminopropyl)triethoxysilane (APTES), ammonium hydroxide (NH₄OH) (28% NH₃ in H₂O, 99.99%), tetraethyl orthosilicate (TEOS) (99.999%), 11-azidoundecyltriethoxysilane (97%), ADIBO-PEG4-acid (90%) were purchased from Sigma Aldrich, Sweden. Ethanol absolute (99.8%) was purchased from VWR, Sweden. Ultra-pure MilliQ water used from MilliQ (IQ 7010) system.

### Fluorophore Stock

Stock solutions of fluorophores were prepared by adding 1 ml of DMSO to 1 mg of respective fluorophore.

### Optically Encoded Nanoparticles Library

Encoded NPs were synthesized by adding either of [0, 0.8, 1.6, 3.2, 6.4] μl Cy3-NHS, Cy5-NHS, Cy7-NHS respectively to 4 μL of APTES solution and raising the temperature to 37° C. during stirring or shaking. The APTES solution was prepared by first adding 10 μl of APTES to 990 μl of EtOH. For example, to produce a nanoparticle type batch of encoding [8, 16, 0], 0.8 μL of Cy3-NHS, 1.6 μL of Cy5-NHS and 0 μL of Cy7-NHS was added. This way any combination can be made using for example 3 colors and 5 levels according to above example. After 10 minutes, 38 μl of H₂O was added followed by EtOH and the temperature was raised to 55° C. The amount of EtOH was set to reach a final reaction volume of 1 mL after the following additions; 54 μl of NH₄OH was added followed by 38 μl of TEOS during vigorous stirring. After 2 hours, the NPs were washed by centrifugation 3 times using EtOH. The NPs were then redispersed in 1 mL EtOH and stored at 4° C. until further use.

The resulting nanoparticles had an average diameter size of 66 nm as measured by SEM and 75 nm as measured by DLS.

In one embodiment, a 7-plex encoded NP system was synthesized using above method with the following encodings: (32:0, 32:8, 32:16, 32:32, 16:32, 8:32, 0:32).

In another embodiment, a 15-plex encoded NP system was synthesized using above method with the following encodings: (8:0:16, 16:0:16, 32:0:16, 0:8:16. 8:8:16, 16:8:16, 32:8:16, 0:16:16, 8:16:16, 16:16:16, 32:16:16, 0:32:16, 8:32:16, 16:32:16, 32:32:16).

### Nanoparticle Coating

NP surface functionalization was performed by adding 100 μL of NPs (175 mM Si) to ethanol (248.5 μL), followed by H₂O (226.5 μL). To this was added ammonium hydroxide solution (20 μL, 2.8%, 1:10 dilution in EtOH). Finally, 11-azidoundecyltriethoxysilane (10 μL, 1:4 dilution in THF) was added and the temperature raised to 37° C. during stirring. After 18 hours, the NPs were washed by centrifugation 3 times using THF. The N3-NPs were then redispersed in 100 μL THF and stored at 4° C. until further use.

Above functionalized N3-NPs (50 μL) were added to H₂O (112.6 μL) followed by DBCO modified oligo (4 μL, 100 μM). The temperature was raised to 37° C. After 1 hour, ADIBO-PEG4-acid (1.1 μL, 420 mM) was added. After 2 hours, the NPs were washed by centrifugation 3 times using EtOH. The NPs were then redispersed in 50 μL EtOH and stored at 4° C. until further use.

In another embodiment, above functionalized N3-NPs (50 μL) were added to H₂O (112.6 μL) followed by DBCO modified oligo (4 μL, 100 μM) and ADIBO-PEG4-acid (0.3 μL, 115 mM). After 5 hours, the NPs were washed by centrifugation 3 times using EtOH. The NPs were then redispersed in 50 μL EtOH and stored at 4° C. until further use.

In another embodiment, NP surface functionalization was performed by adding 100 μL of NPs (175 mM Si) to H₂O (448 μL). To this was added ammonium hydroxide solution (2 μL, 28%) followed by mPEG5k-triethoxysilane (45 μL, 20 mM in H₂O) and N3-PEG5k-triethoxysilane (5 μL, 10 mM in H₂O). The temperature was raised to 75° C. during stirring or shaking. After 18 hours, the NPs were washed by centrifugation 3 times using H₂O. The PEG-NPs were then redispersed in 100 μL H₂O and stored at 4° C. until further use.

In another embodiment, NP surface functionalization was performed by adding 100 μL of NPs (175 mM Si) to H₂O (448 μL). To this was added ammonium hydroxide solution (2 μL, 28%) followed by mPEG2k-triethoxysilane (45 μL, 20 mM in H₂O) and N3-PEG5k-triethoxysilane (5 μL, 10 mM in H₂O). The temperature was raised to 75° C. during stirring or shaking. After 18 hours, the NPs were washed by centrifugation 3 times using H2O. The PEG-NPs were then redispersed in 100 μL H₂O and stored at 4° C. until further use.

Above functionalized PEG-NPs (30 μL) were added to H₂O (5 μL) followed by DBCO modified oligo (5 μL, 100 μM). The temperature was raised to 37° C. After 18 hours, the NPs were washed by centrifugation 3 times using H₂O. The NPs were then redispersed in 300 μL H₂O and stored at 4° C. until further use.

This way, each of the NP type in the 7-plex encoded system could be functionalized with one unique DBCO modified oligo per NP type, to yield a 7-plex NP system that targets 7 different detection targets.

What is particularly surprising is that when a PEG chain of 4000 Da or greater was used stability to the nanoparticle dispersion was provided, but the oligo was unable to bind to the target site. However, when using the shorted PEG4 chain, stability was still provided and the oligo was able to bind to the target site.

### Nanoparticle to RCP Hybridization

14 plex demonstration was achieved by detection for the following common flue pathogens using 7-plex NP library and 2-plex detection oligo library.

| Target ID | Target organism |
|---|---|
| 1 | Influenza Victoria_NP |
| 2 | Influenza Yamagata_NP |
| 3 | mecA AMR gene |
| 4 | OXA-48 AMR gene |
| 5 | E. coli |
| 6 | S. aureus |
| 7 | P. aeruginosa |
| 8 | SARS-CoV-2_ORF1ab |
| 9 | CoV-NL63 |
| 10 | CoV-229E |
| 11 | CoV-OC43 |
| 12 | CoV-HKU1 |
| 13 | Influenza H3N2_NP |
| 14 | Influenza H1N1_NP |

14 different RCP stocks (2 μL) were added together and diluted in SSC 1× buffer (Invitrogen) by the addition of 140 μL SSC 1× buffer to a PCR tube (200 μL). A circle was marked on Superfrost-plus slide (25×75×2 mm, VWR) using a diamond tip pen. Next, 4.5 μL of the diluted RCP solution was added as a drop on the marked circle, followed by drying in a 37° C. oven for 5 minutes. The marked circle with the dried RCPs was washed by pipetting 200 μL PBS-tween20 (0.01M, 0.05% tween20, Invitrogen), the washing step was repeated 2 times and the area surrounding the marked circle was cleaned using a microfiber tissue.

A hybridization chamber (Grace Bio-labs, Secure-seal hybridization chamber 8-9 mm Diameter×0.8 mm depth) was attached over the marked circle on the superfrost-plus slide. Next, the labelling solution was prepared with two detection oligos. 130.6 μL H₂O was added to an Eppendorf tube (1.5 mL), 45 μL SSC buffer (4×, Invitrogen) followed by 10 seconds vortex of the solution at max setting. Next, 1.8 μL AF750-detection oligo (1 μM) and Atto425-detection oligo (1 μM) was added followed by vortex for 10 seconds at max setting. The oligo functionalized nanoparticle stocks prepared above was sonicated for 20 seconds. Oligo functionalized nanoparticle stocks was added (7×2.5 μL) to the Eppendorf tube followed by 5 seconds vortex at max setting, 10 seconds sonication and 10 seconds vortex at max setting.

The prepared labelling solution was added to the hybridization chamber filling the chamber until full (45-50 μL). Adhesive plastic covers (3M VHB) were attached to the holes of the hybridization chamber. Next, the prepared slide was incubated for 1 hour in a oven at 37° C. After incubation, the plastic covers were removed using tweezers and the labelling solution was removed, emptying the hybridization chamber. The sample was then washed using the following procedure. 50 μL PBS-tween20 buffer was added to the hybridization chamber, the chamber was washed by removing and adding the liquid 10 times to the chamber using the pipette. The PBS-tween20 buffer was then discarded. The washing step was repeated 3× times.

Next, the hybridization chamber was detached form the superfrost-plus slide using tweezers. The slide was covered with a carboard box and allowed to dry for 5 minutes in room temperature. Next, 7 μL slowfade (Gold antifade mountant, Invitrogen) was added to the marked circle and covered with coverglass (Menzel-Gläser 24×50 mm #1, 5).

### Image Acquisition

All fluorescent microscopy imaging was performed using a standard epifluorescent microscope (Zeiss Axio Imager.Z2) with an external LED light source (Lumencor SPECTRA X light engine). The microscope setup used a light engine with filter paddles (395/25, 438/29, 470/24, 555/28, 635/22, 730,50). Images were obtained with a sCMOS camera (2048×2048, 16 bit, ORCA-Flash4.0LT Plus, Hamamatsu) using objectives 20× (0.8 NA, air, 420650-9901) and 5× (0.16 NA, air, 420630-9900). The setup used filter cubes for wavelength separation including quad band Chroma 89402 (DAPI, Cy3, Cy5) and quad band Chroma 89403 (Atto425, TexasRed, AlexaFluor750). All samples were mounted on an automatic multi-slide stage (PILine, M-686K011). The nanoparticles were imaged in the Cy3 and Cy5 channel using 100 ms exposure. The detection oligo was imaged in the AF750 channel using 200 ms exposure. The images were obtained using Z-stack with 5 μm height and 0.25 μm slice thickness resulting in 21 slices. All images were taken in ambient, dark microscopic room conditions.

Image processing and signal decoding for a 14-plex detection system with 7-plex nanoparticle library co-labelled with 2-plex molecular probes

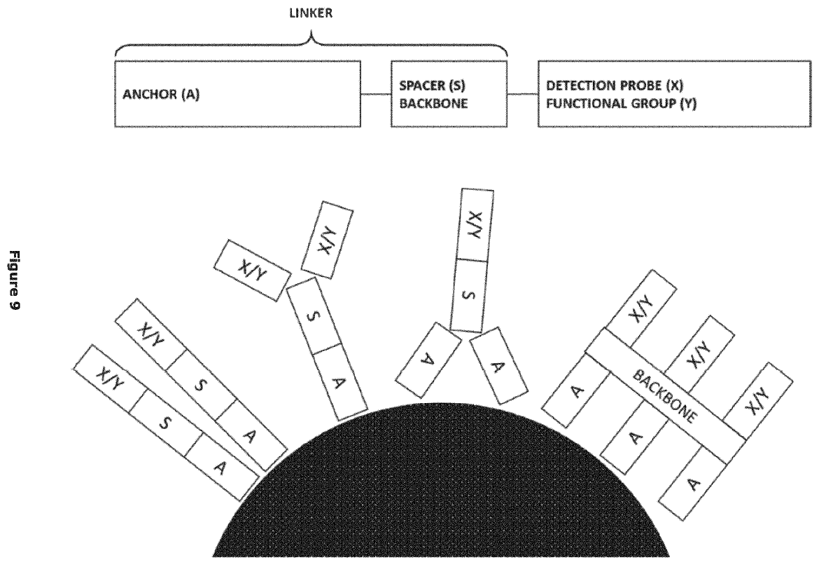

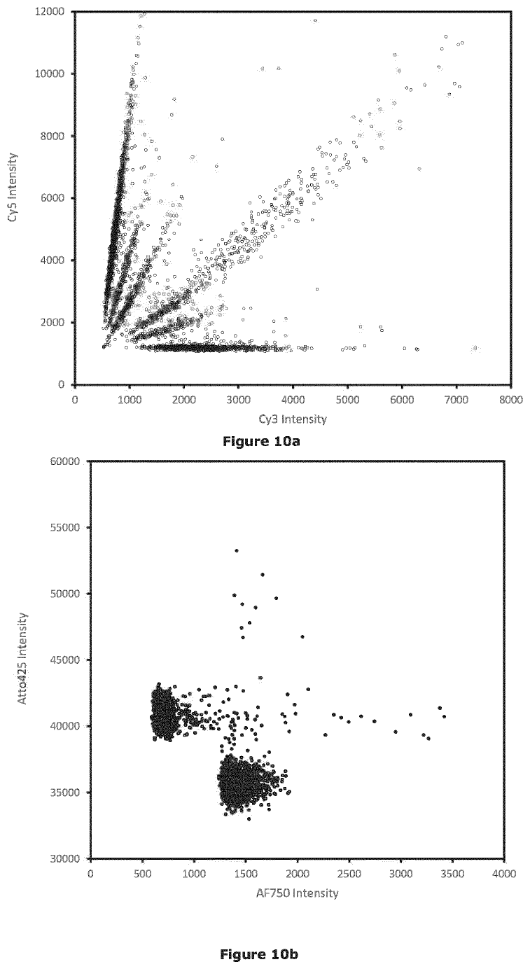

A typical image and signal decoding procedure is shown in FIG. 10. The images were analyzed using ZEN 3.2 (blue edition) software or ImageJ. In one embodiment, orthogonal projection was made using maximum projection method from the Z-stack images. In another embodiment, orthogonal projection was made using average projection method. In another embodiment, no orthogonal projection was made, instead the signal spot was segmented in 3-dimensions and the signal was analyzed in the 3-dimensional space.

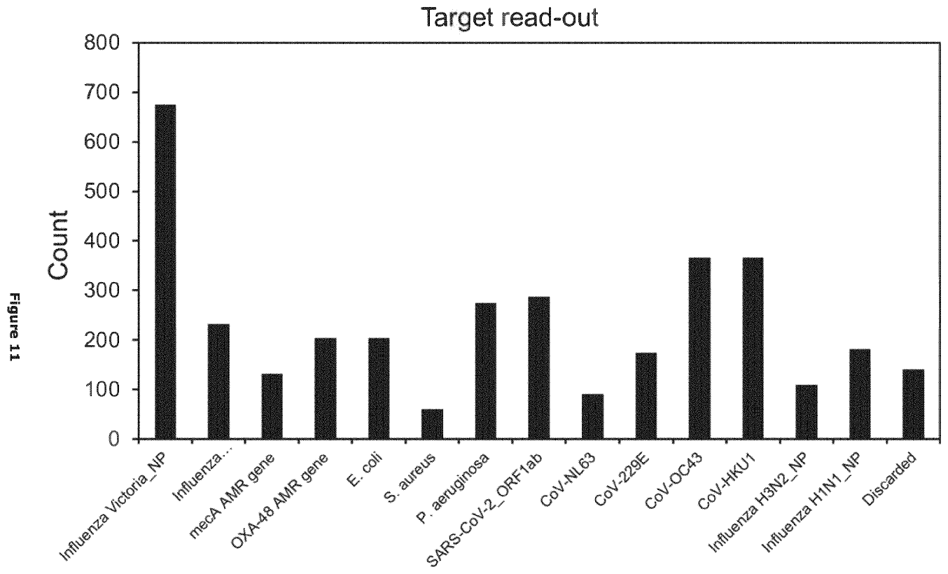

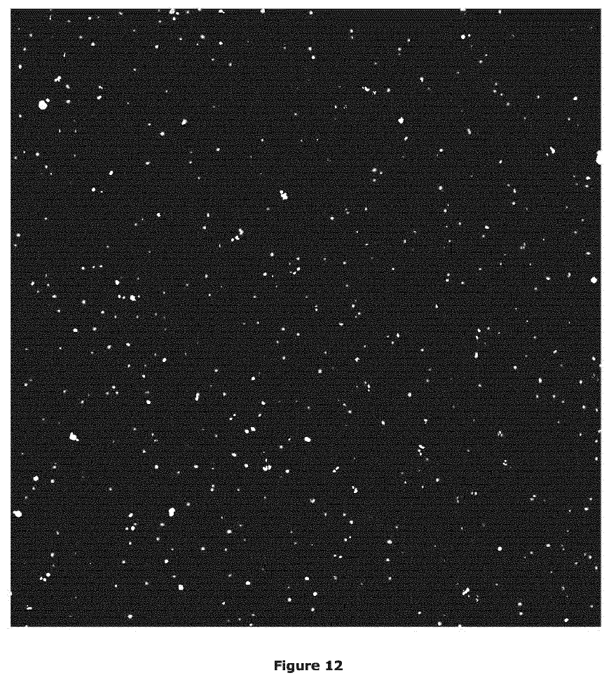

FIG. 12 shows an image from a sub-section of a dataset containing 3500 spots with 14 different target biomolecules (amplified to give RCPs) randomly immobilized onto the microscope slide, and probed with a NP library of 7-plex co-labelled with detection oligo (molecular probe) library of 2-plex. The co-localization between the NP signal and detection oligo signal acts as a method to ensure NP binding to biomolecule and therefore can be used to filter out false positives, in addition to expanding the degree of multiplexing. In this case false positives can be non-specifically bound particles or aggregates of particles.

In one embodiment, to decode the nanoparticle type and detection oligo identity, the fluorescence intensity/profile was measured in respective channel for each spatially resolved signal spot containing a cluster of nanoparticles bound to the biomolecule (RCP). The method of analyzing a single spot and optically decoding the nanoparticle type identity is performed by drawing a line profile over each spot in FIG. 12. The values for each spot in their emission wavelength (separated by the acquisition channels Cy3, Cy5, Atto 425 and AF750) as well as the intensity values from each channel is shown in FIG. 10(a) for the NP emissions and in FIG. 10(b) for the detection oligo emissions from such line profile for all 3500 spots. By identifying each nanoparticle type and detection oligo type identity by their emission identities and setting thresholds thereafter, each of the 3500 spots can be turned into a target call corresponding to the target biomolecule identity according to the method as shown in FIG. 11, thereby showing a multiplexed counting read-out of each target biomolecule in the sample. Any spots that do not fit into the thresholds set for the NP/detection oligo identity can be discarded to remove any wrong or false target calls, also shown in FIG. 11. The following table summarizes the outcome of the results in FIG. 11 and lists the target genes together with the target type that were analyzed using the method.

| | | | Molecule |
|---|---|---|---|
| Target ID | Target organism | Type | Count |
| 1 | Influenza Victoria_NP | RNA influenza virus | 675 |
| 2 | Influenza Yamagata_NP | RNA influenza virus | 232 |
| 3 | mecA AMR gene | Antibiotic | 132 |
| | | resistance marker | |
| 4 | OXA-48 AMR gene | Antibiotic | 204 |
| | | resistance marker | |
| 5 | E. coli | Bacteria | 204 |
| 6 | S. aureus | Bacteria | 60 |
| 7 | P. aeruginosa | Bacteria | 275 |
| 8 | SARS-CoV-2_ORF1ab | RNA corona virus | 287 |
| 9 | CoV-NL63 | RNA corona virus | 92 |
| 10 | CoV-229E | RNA corona virus | 175 |
| 11 | CoV-OC43 | RNA corona virus | 366 |
| 12 | CoV-HKU1 | RNA corona virus | 366 |
| 13 | Influenza H3N2_NP | RNA influenza virus | 110 |
| 14 | Influenza H1N1_NP | RNA influenza virus | 182 |

| | |
|---|---|
| Discarded spots | 140 |
| Analyzed spots | 3360 |

What is particularly surprising is that the shelf-life of the NP library particles was longer than a year, despite regular use and exposure to light, as the emission signature and the probing quality of the nanoparticles did not show any significant change or deterioration over this period. In particular the emission signature of the particles can be expected to be sensitive for long term storage as the fluorophores are naturally bleached over time and light exposure. By encapsulating the fluorophores in the nanoparticle matrix, the fluorophore bleaching was likely slowed down to the extent where high quality read-out could be achieved after 1 year of storage.

In another embodiment, to decode the nanoparticle type and detection oligo identity, instead of drawing a line profile over one spot a circle was drawn around the spot. The max intensity was measured for each channel inside the circle and the background signal was subtracted by averaging the background signal with an equivalent spot placed on an area where there were no signal spots were found. In another embodiment, the background signal was averaged by using the min value inside the circle. In another embodiment, the background signal was averaged by drawing a second circle around the first circle, removing the pixel information from the first circle, and measuring the average intensity in the remaining pixel information from the larger circle.

1. A nanoparticle which comprises;
a. a core comprising a first fluorophore, which is a semiconductor; and
b. which core is coated with a first layer, wherein the first layer comprises a second fluorophore;
wherein, the emission and/or excitation wavelength of the first fluorophore is different to the emission and/excitation wavelength of the second fluorophore.

2. The nanoparticle according to claim 1, wherein the second fluorophore is embedded within a matrix provided by the first layer.

3. The nanoparticle according to claim 1, wherein the first fluorophore is selected from the list consisting of quantum dots, rods, semiconductor quantum dots, perovskite quantum dots, quantum rods, Pdots and silicon quantum dots, or mixtures thereof.

4. The nanoparticle according to claim 1, wherein the second fluorophore is an organic fluorophore, optionally wherein the organic fluorophore is selected from the list consisting of Atto 425, Alexa fluor 405, Alexa Fluor 488, fluoresceine, DiO, Atto 488, BODIPY FL, Cy3, DiI, Alexa fluor 546, Atto 550, BODIPY TMR-X, Cy5, Alexa fluor 647, Texas red, DiD, Atto647(N), Atto 655, Cy7, Alexa fluor 680, Alexa fluor 750, Atto 680, and Atto 700, BODIPY, Brilliant Violet, Cyanine, Alexa, Atto, fluorescein, coumarin, rhodamine, xanthene fluorophore families and derivatives and combinations thereof.

5. The nanoparticle according to claim 1, wherein the nanoparticle comprises an outer layer, wherein the outer layer comprises a metal, an inorganic oxide or a polymer.

6. The nanoparticle according to claim 1, wherein the nanoparticle comprises a coating, wherein the coating comprises:
a. a repulsive component; and/or
b. a linker; and/or
c. a detection probe.

7. The nanoparticle according to claim 1, nanoparticles have an average diameter of less than about 300 nm.

8. (canceled)

9. A method for multiplexed detection of a plurality of target biomolecules using optical encoding using a plurality of nanoparticles according to claim 1.

10. A method according to claim 9, wherein each target biomolecule has at least one detection target, comprising the steps of:
a) providing a plurality of nanoparticle types comprising a plurality of nanoparticles according to any one of claims 1 to 8, each nanoparticle having a coating that provides binding affinity of the nanoparticle to a type-specific detection target, wherein the combination of the first and second fluorophores in the nanoparticles of each nanoparticle type generate a signal which is unique for that nanoparticle type;
b) providing a sample comprising a plurality of target biomolecules;
c) contacting the sample with the plurality of nanoparticle types, thereby allowing the nanoparticles to bind with the detection targets of the target biomolecules; and
d) optically decoding the fluorophore signals emitted by the nanoparticle of the nanoparticle types bound to the detection target of the target biomolecules by measuring the wavelength and intensity of the emitted signals, thereby detecting the presence and identity of the target biomolecules.

11. The method according to claim 10, wherein prior to step c), the target biomolecules are prepared by binding them to at least one molecule comprising the detection target, such as a barcoded nucleic acid molecule, padlock probe or initiator sequence for subsequent amplification.

12. The method according to claim 10, wherein the detection target comprises a nucleic acid molecule, which is, or facilitates a molecule that is, amplified using RCA or multiple hybridization events.

13. The method according to claim 10, wherein the decoding is effected by optical decoding such as by optical imaging.

14. The method according to claim 10, further providing one or more molecular probes, wherein each molecular probe comprises a fluorophore that is bound to a nucleic acid molecule, an antigen or an antibody providing binding affinity of the molecular probe to the specific detection target.

15. Kit-of-parts, comprising, in separate containers,
(i) a plurality of nanoparticles according to claim 1;
(ii) a probing buffer, comprising a solution with controlled pH, salt concentration and additives facilitating specific detection target binding of the nanoparticle(s); and
(iii) instructions for use of the kit in a method comprising the steps of:
a) providing a plurality of nanoparticle types comprising a plurality of nanoparticles according to any one of claims 1 to 8, each nanoparticle having a coating that provides binding affinity of the nanoparticle to a type-specific detection target, wherein the combination of the first and second fluorophores in the nanoparticles of each nanoparticle type generate a signal which is unique for that nanoparticle type;
b) providing a sample comprising a plurality of target biomolecules;
c) contacting the sample with the plurality of nanoparticle types, thereby allowing the nanoparticles to bind with the detection targets of the target biomolecules; and
d) optically decoding the fluorophore signals emitted by the nanoparticle of the nanoparticle types bound to the detection target of the target biomolecules by measuring the wavelength and intensity of the emitted signals, thereby detecting the presence and identity of the target biomolecules.

16. The kit-of-parts according to claim 15, wherein the kit comprises a plurality of nanoparticle types each comprising the plurality of nanoparticles, wherein the combination of the first and second fluorophores in the nanoparticles of each nanoparticle type generate a signal which is unique for that nanoparticle type.
